SEQUENCE LISTING (56 sequences)

SEQ ID NO: 1 - DNA, 1327 bp, Arabidopsis thaliana
gagccatatatatgatgctcattgtgtttgttcttatgtaactactcttgcaactctaagttcaaagtgtcaaatcaagattcaagatcatcatcataataaaatatcaaatcacaaacttagaatctcttacacaaacatacaaatagagataacagtaatctttcctcatctattcatcacaaccatatattatccatataataaaaactactaaaaccgaatcgagacaaaaggatcctcatgatctcataatctatagctataacataacatagcaaatatataatcatcataatgactatatattattaagatcaagaatcaagatgtgatcttaattatatcttaacaataagcaatacactccttcttacaatccatagtgaaagtcttaaaaggcttaacaatgattaatgtttgccattttaatctcccttgaccgagttttttcatgttgagtctatatactttaataactaatttatagccaaattaacataatgtggcgaatcatgtaatgtacgtgaaaacgtaattctgttttaagcaaaatttgcacatatacattacgattgtttgatttatcatataatttttgattctgtattttgttaaatagttagttatatattaagcaaagattgcacacattacgattctttgattgccatataattagtttcatcgtactacctttggaatattccactatctatcaaagagattcaactatccgtggtcaccattttataatctataaagtataaagtgtgtaaaaaaaacaaattcaaaacgatatacacattaaaaaaaaatccggaattggtttgctgtcctgtgatcctatatttcggtgtagagtcttctatatttcaaaagttcagaatataatcattctatactaaattgagtaattcagtcaatcatgatctaccaacttcttaattacagttacctaacctactcatttagttagaaattattgatatcctcttatagtcttatactcatttgaattataattaggtaatatatataattaggtacactattcgtatatctataataagaaagacgacaattgtaagagttaaaactgagccaaaaagttatggtgggaatatcagtaacgctacacgagagataaaaccggtctgattcggaattaccataataagttgaataaaccaataattgaatccgaaccaaattcgaatctaaccccaaattttattgcttaagacgaattatttactatttatatgtatataaaaaagcttctataccacacagtcacacatgcacacacttctcacttcagaca

SEQ ID NO: 2 - DNA, 1326 bp, Arabidopsis thaliana
agccatatatatgatgctcattgtgtttgttcttatgtaactactcttgcaactctaagttcaaagtgtcaaatcaagattcaagatcatcatcataataaaatatcaaatcacaaacttagaatctcttacacaaacatacaaatagagataacagtaatctttcctcatctattcatcacaaccatatattatccatataataaaaactactaaaaccgaatcgagacaaaaggatctccatgatctcataatctatagctataacataacatagcaaatatataatcatcataatgactatatattattaagatcaagaatcaagatgtgatcttaattatatcttaacaataagcaatacactccttcttacaatccatagtgaaagtcttaaaaggcttaacaatgattaatgtttgccattttaatctcccttgaccgagttttttcatgttgagtctatatactttaataactaatttatagccaaattaacataatgtggcgaatcatgtaatgtacgtgaaaacgtaattctgttttaagcaaaatttgcacatatacattacgattgtttgatttatcatataatttttgattctgtattttgttaaatagttagttatatattaagcaaagattgcacacattacgattctttgattgccatataattagtttcatcgtactacctttggaatattccactatctatcaaagagattcaactatccgtggtcaccattttataatctataaagtataaagtgtgtaaaaaaaacaaattcaaaacgatatacacattaaaaaaaaatccggaattggtttgctgtcctgtgatcctatatttcggtgtagagtcttctatatttcaaaagttcagaatataatcattctatactaaattgagtaattcagtcaatcatgatctaccaacttcttaattacagttacctaacctactcatttagttagaaattattgatatcctcttatagtcttatactcatttgaattataattaggtaatatatataattaggtacactattcgtatatctataataagaaagacgacaattgtaagagttaaaactgagccaaaaagttatggtgggaatatcagtaacgctacacgagagataaaaccggtctgattcggaattaccataataagttgaataaaccaataattgaatccgaaccaaattcgaatctaaccccaaattttattgcttaagacgaattatttactatttatatgtatataaaaaagcttctataccacacagtcacacacgcacacacttctcacttcagaca

SEQ ID NO: 3 - DNA, 2018 bp, Arabidopsis thaliana
gtagtgaactacgatatatatcattgtggactgacttgtggtgtgtgctgtctcagcgattagcaacctcacaaataaagttaatactaataagtaccctactgtttaacgacctcacaaatcaatactaataacttctaaatttgaaatttgttctctacgtttcacactacatttatggataatcgggtgtatctatagtatatgcatgcgttcgtatgagttttaataccagcgttgactgtcggcaagtaggaaataatccaattaataatacgtttgacaaaagattaaactgtagtactatatataatggaatatttaatccagatatcaaccgttgaaagttatctaatttaatttgataacgatttccaggactgtccccaaatctatctgaaagttattaatcactcctttctaaacaataattgaactttttcttaaaaaaacttctacgacaacacatttcctttgcataacgtagaagtcaatcaaagtttttaaatacttctatcaaatttttaagtaaaatagtattgacacgaaatgcaaaagacgaagtatactgaatataaaatatcacggctacaatgcaacatttaagaattagatgattggaaatcgatacagaaaaataatctaagagaattaggccgtcacttgtgttgtgtgggagcaaaacaaggaccaaaaatatcgggacaaataggttggtccaacctataggtagaggtagcccacttggcatagctcataataccattaccagctcatatgttttttcaaggattggagaaaattaaagaaagatgtaatcgattagagtaacagtggagtgctgaatttaagttagttaagaaaataattggtgttacttcttataaacttttaactcaaaaccaattcgtaatgaatagatagatccatgtctattatatcttatatactattcaaacctcttcttatatatttttccaatgtggattattcgcccatagataaaagataaaacttaacaattggtaagacaatatgacataaagtccttagttctacttacaaagaattttgtcaattaccttccaaaatttagatcttctaaaccctaagttattgggtttcaccaatataatgggtcatttcatctattcacccgaccgttagatttaccaatttctcatcatatctcgattttcaacatttaagaaagtaatcaagtttagccgaaatgcaagatgatacagaaacaatagcgtttaacggtgttagatgataaactcatcaactccattaagaaaaccaatcctgtaagaggtaaagaaggggagaccataattaatgtctaatactttcgtaatgaccactattaatgattagtactatgatctatgaagttgaagctctcttttttttttttttttttcccttcacgtccatagttagttacagcattgatgaaatttttgctgagaatagacgaccctttatcctccaccctacgctttaagtggttgggagttagaccctgccagatagattccaatcctaagataagtctgtttaacaaacctatcatatgtgaaagtgaaaaccattatgttgaagaattatctaaggcgtagagataatttctgcagcaaaaacatttttttaaacattgcgttatacattttaggatagtttatataatcagccaaagtgtatatttctgtaaaacacattactatcttgacatttttgtgataagctatataatcagtaacctgctacgtatagcttaaccccactattataattatgattcctcattcagtaaaactatatagctgaattaataaagtttattagggtctaatgaagttggtgtgatcatttaataatattgttatttcataactcggaattgaattatttattacccttgccatcttaaatctacatttgcaactcacccaaaagctttatcctttgtgttttttccactgtatactgaaaacaaatctgaggtgacgaag

SEQ ID NO: 4 - DNA, 1974 bp, Arabidopsis thaliana
atacaaaaatattttatagtagtgaactacgatatatatcattgtggactgacttgtggtgtgtgctgtctcagcgattagcaacctcacaaataaagttaatactaataagtaccctactgtttaacgacctcacaaatcaatactaataacttctaaatttgaaatttgttctctacgtttcacactacatttatggataatcgggtgtatctatagtatatgcatgcgttcgtatgagttttaataccagcgttgactgtcggcaagtaggaaataatccaattaataatacgtttgacaaaagattaaactgtagtactatatataatggaatatttaatccagatatcaaccgttgaaagttatctaatttaatttgataacgatttccaggactgtccccaaatctatctgaaagttattaatcactcctttctaaacaataattgaactttttcttaaaaaaacttctacgacaacacatttcctttgcataacgtagaagtcaatcaaagtttttaaatacttctatcaaatttttaagtaaaatagtattgacacgaaatgcaaaagacgaagtatactgaatataaaatatcacggctacaatgcaacatttaagaattagatgattggaaatcgatacagaaaaataatctaagagaattaggccgtcacttgtgttgtgtgggagcaaaacaaggaccaaaaatatcgggacaaataggttggtccaacctataggtagaggtagcccacttggcatagctcataataccattaccagctcatatgttttttcaaggattggagaaaattaaagaaagatgtaatcgattagagtaacagtggagtgctgaatttaagttagttaagaaaataattggtgttacttcttataaacttttaactcaaaaccaattcgtaatgaatagatagatccatgtctattatatcttatatactattcaaacctcttcttatatatttttccaatgtggattattcgcccatagataaaagataaaacttaacaattggtaagacaatatgacataaagtccttagttctacttacaaagaattttgtcaattaccttccaaaatttagatcttctaaaccctaagttattgggtttcaccaatataatgggtcatttcatctattcacccgaccgttagatttaccaatttctcatcatatctcgattttcaacatttaagaaagtaatcaagtttagccgaaatgcaagatgatacagaaacaatagcgtttaacggtgttagatgataaactcatcaactccattaagaaaaccaatcctgtaagaggtaaagaaggggagaccataattaatgtctaatactttcgtaatgaccactattaatgattagtactatgatctatgaagttgaagctctctttttttttttttttttttcccttcacgtccatagttagttacagcattgatgaaatttttgctgagaatagacgaccctttatcctccaccctacgctttaagtggttgggagttagaccctgccagatagattccaatcctaagataagtctgtttaacaaacctatcatatgtgaaagtgaaaaccattatgttgaagaattatctaaggcgtagagataatttctgcagcaaaaacatttttttaaacattgcgttatacattttaggatagtttatataatcagccaaagtgtatatttctgtaaaacacattactatcttgacatttttgtgataagctatataatcagtaacctgctacgtatagcttaaccccactattataattatgattcctcattcagtaaaactatatagctgaattaataaagtttattagggtctaatgaagttggtgtgatcatttaataatattgttatttcataactcggaattgaattatttattacccttgccatcttaaatctacatttgcaactcac

SEQ ID NO: 5 - DNA, 490 bp, Arabidopsis thaliana
tcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttcttcattatattttgttaagtccactcttctctctcatatcttctaaccaaaacagagtcacaaggggctcttaagcccttccaactaaattcttttcttttgttctcttgaaactgaatccaccagacaaaa

SEQ ID NO: 6 - DNA, 2255 bp, Arabidopsis thaliana
tgggttttatttttgacatttggttttatactttagttccgttgactttcgcctccaccataatttctccaattcagatttgattcggtctgaacacaaagtccggtttggtttcttatttgtcttaatatcgattactttccatctataaaatatttttctacaacatcttaagaattataattgagtgatgttgatgctactattttaagtttagaaaataaacactaaaaagacaaatgtctcactcatcaaagtaaaactcttgaaaagtgcaagagctctgaaatttgagaacgaagacaagactccttgtttttttttgtttttttttgctaaaaatttaaatattcattattacaatgaaaatttcggttacataataaatggtaaccaaatcatggttccatgacaaaaaaggataaaaagcatggaagcataccaagactccttgttactacgtcaatctcttttatacgttttcagccaagattccggattatgaaagaatcttgggattctaacactttttctttttttgcttgaaagaggtttacaaattttaacactttttttttgttgaggattttagagtgaaacacatgttttgaactgtcttcaactgaacaattcatgttaggcgtctatataaccgtcggttattcacgaggtaactacacatgaacatgataaatttactctctcttttcattaaaaaaaagttgtacaacttaattacttatgtcatgaaaatagtatatacgtaaaagtagattatttttgtggttttccttttttttactataacaataaataattctatgttacctaaattttcttaggtagtataatggatcaaattgatatggagtaaacaaaagaaaaacttaaataatctggtctataatttgaagcgcttcaagccttcaacatcaatccgagtacgaacaataatatgagatttcatcaaaatattatcctggaaacgatttttcatttatatgcgattatattgttaatgaaagttggaaatacataatctagacacgtaaatgtcgtattgatcatgttgtgaaatgagctgtcgccttggtggcactttttggcattctctatttctctttccacatttaccacaatgtatccaaataggcaaatatataagcttagagagttggctgcacgtttttgctaaacttgataaatgagtcaatacaaccaatatagccaccatccatatctacaaatctacacttatcatctaaacttgaagaatatttgttattttatcactaaccacaaaagacaagactcgttacttaagttaaatgatagtgacatgattaagagaatattagctattaggtcggaaataagagaaataagactggtagtggtatggttatgtaaattatcagtacatgtatataacacttgtccaaataatggctttcacattacaagtcattctttccctgagactactgcaagaaacaaacacggaattctcgtgataaacggattagtacgaaggaaaaagtaaaatgcagtaaccaatttttatatttcaaaaaacaaggcattttggatgcaatgaaatatttagatatataaatttgactagtgacaacaatttaaagttgttagatttctcaaatccaaaaaaaaggaaataaataaataaatagtttatggctattcaaattgtgtattattttttctattggttaaaatctataaaagattttttttttattacttcttaaatttatgtttatagccaaaacatctaataaaatgggacagagaataataactaggaattcaaacacattatcaatgattagcagaataaaagtttggaacatctaaacctaatgactttatacttcccctttttagagtttactttgtatggaaaactttgtaagctaacaaacaaaagtattgaaatcgtgaaaaatagtaaagctttttgagctgcaatatttgatgcgttgaaacgagttggaaacagctttcactacactaaaaacaaacttaatctcaaaatttagatggattaaactcaaaactttttaattaattgaataggattttaggatgatgcagtgaatatagactatttggtgaaaaaatacaacgtaacgtacgtggctgctctaagcctatataacatagcccaagagagtcgtgttctaatgtgattaagtaaagtgagggagaagcaacgagagatagagatagagagatca

SEQ ID NO: 7 - DNA, 1185 bp, Arabidopsis thaliana
ttctctctagcaaaactctctctctttctcccttgtagaattaattagctatcataaatatagtagttcatcagttccacttccactaaattattgtttttggcaaaacagtaacttaagttatataaaaaaaaaaatcattagtcaatcaatcacagtcctttatgataaaacgaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaatgaatttaatatattctaagtaacttgcttaaattttttttcaaactctaaagacataactaacataaagtaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcactcaacacaaactctttacgaatacttttaaggc

SEQ ID NO: 8 - DNA, 2119 bp, Arabidopsis thaliana
cagaatatctaaccatttcatccagattatatatttgttaatatctaacattatcgatattctatcgcaacatggaatcattaatatctaacaatttcgaacattttcaatgttcataacgcaaaacaatgtcaaagtaaattcaaactacacgaagtaaatgtattgtatgaccacatatacaaagtataggacgtcatgtggttaacaccatagacatacaattccgataaaccggtcagttgactccggcgttgactagggttgaccggcgttgaccaacaaaaaaattcaaaaaaatcttttaaattattttaaatattcaaaaatacaaaatattttttttttggttttgtatattcaaaaacatattctatatttcaatgcattaaatcttagaaaaattagttttacaaaaaaaatcaaaatttaactaaaaatagattaaaaatcattattaaattttaaattttaaatgaaaacaggaaaatattattatagttaattaagtaaggaaattgcttatttttatagtgtcaattaaaacacttcaattatttctatacaatattttttataaaaaaaaatcaaccacaaaaattattagaataaaacgtaatacaaatgaattttattttaaaaacttttttgctgaaatcaacattgttagattttctatctttttatatattaaaaagaaaaattgcaagtttttggttgtttatgtgttactacgagaacttttcttaataatatttgttacaaaaggaactacatagtatacaaaaataaatttagactaaagagtatataaaaaatattataattttctttaccatgcaaactttagattaaagagtcatatactcaatttcatattgcttcctaatacaattgagtatatgactctttaatctaaagtttaataatgatttttattctagttttagtttagttttgaaattaaaaataaaactaattattataagatttaatgcattgaaaatacaaatatatttttacgaaatatagaatatgtttttgaatatataaaagaaaaaaaatattttcgtatttttgagtattaaaaataattttaaatttttttgttggtcaacgccggtcaacactagtcaaagcctgagtcaactgaccggtttaccggaattgtatgtcaatggtgttaaccacatgacgtcctatacttcatatatgtggtcatgtaatacatctacttcgtgtctacttcgtgtagctggatatacaatgtatagtaggtatgtgtgaccatgtattctcttatactttgtttacctagcaatcttttttttaaattaaaataaatatgcggtttagatatgaaactacccaacaaatttaacattttaaacgttcataacgtaaaacgacgtcgttatagacacatattttccatgtgtctgctgacttatcatcttcacggagttgactaacacccgttactttgactctgaattttgtactttttcttaagttgaggtatgaaattcaaataaatatgcggttaatatatgaaaatacccaacaaatttttttggatacgaaaatacactcagaaaatagtacgggtatgaaaatacccttttcccgtatttgatacatgtctaattcggttcaaataaaccgaatatgaaaattttcagttttatttcggaagttaaataaatctagataaccgacctgaaaaacccgagtcccgaccgaaccgaaccgaaattaaattcggtttaattcggaagcatttccaaaaaccgaaattccctaaaaccgaataacccgacccgattaaaccgatttgccgaactcccaggcctaaattcacacttggcttagaaaaactctttgtagatgttaaaattcggtaaaattaacctcaccaaagctaattattaccaggtgaagaaagcattaaaatttcaaagtgtgtatgacagaggttttagaaagcgactgatgtacggacatatcaacaactcccctataaagatactcagctaaacacaaaaacagaatctattctcaacacaacactaaagacaattgtaccaaccacacaaccacaagagagagaaaagtgacc

SEQ ID NO: 9 - DNA, 853 bp, Arabidopsis thaliana
tggttctgctacatgcagatgatactatccgttgttgaatttgtcgattagaattctttttggtgtacacaatgcggttgtcataacgccttaatagcttgtattagtcaaagaactgcatatggtcttgtgttttcttgtcatcgtgtttttgtaaccacaaactgttttgagctatactactatatatattgagatatatctgccgtttcgatacacacttgggatctggggatgagcacatcgtaaaacaaaatagaagttgatcctcaaaacttctttgtaaccttgtgtcatcacaacaaaaaatcttcaatgtgtttgttctctccttaaagtatatcttgattcatgcagtaacaaaggcaaaactcttttgcaagagtatagaaaccagactcaagctgtgcgatggtgattcttttggagaagttggatttgtgctctgatgtaaagggaaacttaagctaaaaggtccatcaatggaggtgacacatagttttagaaaatgtgcttttctcatgctagaaatgttatggagacccaaaaatgcttttcggaaaaaattctcatgctagtagctaggctctacttaacgaggtgacagctaaaataagttctttttattccattttcagaatagtgacattcttctcacaaatatagaaaaactacaattaatgctactgcagagtctgattacgttttaagctaatttttccatttttaggacgtggtagattgtgtagattattgctaaacagctcatgagttcaataattcacttattcttcactccatcttcagcaaaaaaaaaaaaagtaagaagaaacactgaaagctctccactacc

SEQ ID NO: 10 - DNA, 4755 bp, Arabidopsis thaliana
aattcgatagacgctgggtaaaaaaattcggaggacgacgaaagagaaaacgagtgtttcagtcactgccccacggagctctcggaaatttgtcttccccttgtcgtcgtctccctatctactgcttcttcttcgttttcgtcttctttatcaaggtgcgctttagcttctcaacgccgtttgatttttagaatttcgatttttttttttttttcttctagttcttgaatcaatccggaatttggcgactatgttgcttcgtttgtaaatcgtattctcctgtttagaaatcttcaattgactgtgttataggaacaatttaaatctcaatttcaatgtctcttttagtcaccttcgtgtagtaatttgcttttgaattactgttaatgaatctcaaaaaatggattttataatttgggaaaaggggcttctgggtttaattaaagaacacgagataaggtctggttttttcttttcatttctttgtgtgtgtttttggtttctttgattttcttctgggttatggtccgtttgagtctggtgatagttagttggcaaccaatttttattgatctattacaatcgagaacacaaaactaaaccctaagaaagaagtacataaagttgttgaaaagatctcgttaactctcccaaagtcctagggctttcacacaaccagtgattaaataacctttgagctgttctccttcccacactttatatgtgtgtttgtggtttgtctaatttgtgaggagcttctatgaaacctctggttattttaattgttttctgcaattcctgattgatatgtttatatatatttcttgtatttgtgaatttgtgtaggaatgctgtttaattggaatcaacaatggagaatttgacggaaatagaatcaacgatggagagtttaacggaaatggagagtgagagagttgaacagggtaccgataaggaaattggaagtggagagaaaaggcaggatgatgtaaaggaaacggagaatgagaattctggagagagagtaggagaggaagctcctgtcagggaacatgaagattctccatgtctcattgttattgaagaaggtacttccctagcttcccttgaggaggtgaccaatgctgatgatctgccgaagattgatgatgagaagaattcccaatttgaaacaagcccgcatccaagtccttctccttcagtagctttagacactgaagaagggttaatcaaccctactgcagaagacactgtagaagagaacatagtgtctagcgaagtaagttcggatatcttgaaagatgacggagatgccgtcgaggttgacagagatactgcagaagtccaggaagaaacggccaacatacctgaatccaaactctcggaggacacaggatcacctcatcatcatgctgatattctgatggtgcaggaaaaagctgcagaagaacatgacatgatagcctctggagaccatgaagaatttccagtcaatcctgataacaaacactctgaagaaaatcagtcaccacatcatcatgctaataatgtgatggagcaggaccaagctgcagaagaacgtgagatcatatccccaggagaacataaggaaattccagccaatcctgatactaaagttgttgaggagaacaatgacaggatagatgagggtgaggctaacaatttgaatttggctggcgatggaagtggagcagtcgatcatgattacttgaccaaaacggagctggacaaagtgctagaggtgcctggttctgagaccatatcaaaactggaggataggccatctgagcatctctcagaaacctcaatgaacgtggaaaaagaactagaaatgcctgccgttgaaattttgccagacaatgacaaaaactctgatgtgttggcagttggagtttctggagacagtgacaatgtggtatctgtcttgcccgcttcccaaacttcctctgatcgtgatgaaggaatgattacagttgatgctgaacctacggaagacatgaaacttgatgttccagattctaaattggttactgatactactgttgactctactaataacaaggatgcccatgttgaggctaatactgaaaggcaagataattctagtgcacttgtgctaaatgatgcaaataatgaaagtgcaccagtgaaacgtgtacctggtccttatgttgcatcttccaatataaagtctgaagcgcggggtagtggagatttgaacaatggagtacataaaatagttcggaccccacctgtctttgatgggaccatgcgcgcaaagcgctctttcctcttggatgatgcgtctgatggtaatgaatctggaacggaagaggatcaatctgcttttatgaaagaattggatagtttttttagagagcgaaacatggatttcaaacctccaaaattttacggggagggactgaactgcctcaagtaagcttgatacccatcattatttggtcactttactgtgttacattttaaaattttcagcaggagctgatatctaatcaatttctttggcacaaggttgtggagagctgtaactagattgggcggatatgacaaggtacgggtcactgtgaatacgcctgttgaatgtcacagcatcttttttgacaagcaaatgtgacttcggcttttcatcttttgttccatcctggcttacttgcatgcgtactgttgttcatgatctagcagtggtgcttttggtgattttctatgattattatatgctttttatactggataggttactggaagcaaattatggcggcaagtgggagagtctttcaggcccccaaagtaagaagaatgcttttcttattagtggtttgtcttagaaattttgggaaatcatgtggatatttttaagaattaccctctaattggtcaattgtttgttcaggacatgtacaacagtatcatggactttccgaggtttctacgaaaaggtgagactatattcaccaccttttcctctctctgcttttggttcgtctatgtgacttttgtatacactggcatgggactgggactctatgtatcaacccttctgagaaataattgaaatgattgaacagtgaacaactgtgaatcatcttgagatatgttttccttaagatacagtaacatcttgtaacattatagtttcttcatttttcaggctcttcttgaatatgagcggcataaagttagtgaaggtgaacttcagataccccttccgttggaactagaaccgatgaatattgataatcaggtaaaattgagaaaaccatatcatgtgtctgtagtttttgtttgatcttcttcttctgattaatgtcagtgttttaacttaacccactgccttgtttctacactaggcgtctggatcagggagagcaaggagagatgcagcatcacgtgctatgcaaggttggcattcacagcgtcttaatggtaacggtgaagttagtgaccctgcaatcaaggtccggtagaatctttttatatgtttcattttacattcacactagatctctcgttttttttttgtcaaacatttaatctatatctcatagtctgaacgaacatactgttttgtaattaataggataagaacttagttcttcatcaaaagcgcgaaaaacagattggaaccacccctggtatgagttctgtttgatgaagaagtgttgttctcatttttattttgaaactttgacatgggttatcacttacatctcacaatgtcatcaggtttgctcaaacgtaagagggctgctgaacatggtgcaaaaaatgccatccatgtatctaaatctatgtacgatttttggctttgtggtctggttttcaatgcgtgataattcacatttgaattctgattccagttgttgtttttcctaggttggatgtgactgttgttgatgttggaccaccagctgactgggtgaagattaacgtacagagaacggtaaaatcaattgccactttcttaaaaacctgagcaatcactttctggttttacatatattaataaactcttccactatctgcagcaagattgctttgaggtgtatgcattagtcccaggattagtccgtgaagaggtaagctctcaaatctcgttgtgtttacatatggatcctaagattgagtttagcactcagtttttgtcttggcaacaataatacaggtccgagtccaatcagatccggctgggcggttagtaataagtggcgaacccgagaaccctatgaatccttggggagctactcctttcaaaaaggtaaatgctggttacatgatttttcagcttacacgtagaatgttgaatgacattttcaaacctccattgaaactgcaggtggtaagtttaccaacgagaatcgatccgcatcacacatcggctgtggtaaccctaaacgggcagttatttgttcgtgtgcctctggagcaattggagtagaaacatttacagtttaacaaagcctttgaagatctgaaagagagaagattgttagaagtagttgttgagagtattttgtttgtatattatgagagattaagcacaacatgagaagagcctttaggaatccttaattaggccatctagtttttattgtctctcctctctttgattagattcttcttctaagtgtcatcactattgatttgttgtagcaccaaacttctttaaacctttctattaagaacacacaaatctacaacctttttatttttttt

SEQ ID NO: 11 - DNA, 810 bp, Arabidopsis thaliana
atgaaatcgttttgcaagttggagtatgatcaagtgtttggcaaagaaaataattcattctcatttctaaaccactcatcactttactctcatcaaagcgagttagcaaatcctttcttcgagttggaagacgagatgcttccttctgctacctctagtaattgttttacttctgcctcaagctttctggctttacctgatcttgaacccatctccattgtgtctcatgaagcagatatacttagtgtgtatggttctgcttcatggaccgcagaagagacgatgttcgtttctgattttgcgaaaaagagtgaaaccacaactaccaagaagaggagatgcagagaagaatgtttttctagttgttctgtttcaaagacattgtcgaaggaaaccatctcattgtacttttacatgccgataactcaagcggctagagagcttaacattggtttaactcttttgaagaagagatgccgcgaattgggtattaaacgttggcctcatcgtaagctcatgagcctacaaaaactcatcagcaatgtcaaggagctagagaagatggaaggggaagaaaatgaagataagctaagaaacgctttggaaaagctcgagaaggagaagaaaacgattgagaagttaccagatttgaagtttgaggataagacaaagagattgagacaagcttgtttcaaggctaaccataagaggaagagaagaagtggcatgtccacgcccatcacatcatcatcttcttctgcttctgcttcttcttcttcttactcttctgtttcgggttttgagagataa

SEQ ID NO: 12 - protein, 269 aa, Arabidopsis thaliana
MKSFCKLEYDQVFGKENNSFSFLNHSSLYSHQSELANPFFELEDEMLPSATSSNCFTSASSFLALPDLEPISIVSHEADILSVYGSASWTAEETMFVSDFAKKSETTTTKKRRCREECFSSCSVSKTLSKETISLYFYMPITQAARELNIGLTLLKKRCRELGIKRWPHRKLMSLQKLISNVKELEKMEGEENEDKLRNALEKLEKEKKTIEKLPDLKFEDKTKRLRQACFKANHKRKRRSGMSTPITSSSSSASASSSSYSSVSGFER

SEQ ID NO: 13 - DNA, 897 bp, Arabidopsis thaliana
atggctgatcacacaaccaaagaacagaagtcattctcattcctagctcattctccatcctttgatcacagctccttaagttatcctttattcgactgggaagaagatcttcttgctctccaagaaaactctggctctcaagcatttccttttactacaacttctctgcctttacctgatcttgaacccttgtctgaagatgtactcaattcatacagctctgcgtcatggaacgaaacagagcaaaacagaggagatggcgcttcatcggagaagaagagggaaaatggaacagtgaaagagacaactaagaagaggaaaatcaatgagagacacagagaacatagcgtgagaatcatcagcgatattactacctacacaactagttcagctccaacgacattgtcaaaggaaactgtctctcgctacttctacatgcccataactcaggctgcaatagcacttaacgttggtttaactctactaaaaaggagatgtcgcgaattgggtattcgccgatggcctcatcgtaaacttatgagcttaaacactttgatcagtaacgtcaaggagctgcagaagatggaaggcgaagagaatgcagaaaaactgcaggacgcgttggagatgcttgagaaggagaagaggacaattgaggatttgccggatttggagtttaaggacaagacaaagaggctaagacaagcttgtttcaaggctaaccacaagaggaagaagaagagaagtctcaagtccgatcagtctcaagtaccctcgtgttcaagcagcggatcagttcctagtgatgagtcggttgatgaagcaggaatggagagtgatgaagaaatgaagtatctcttgtgtggtttctcaagtgaatttactagtggtttgtga

SEQ ID NO: 14 - protein, 298 aa, Arabidopsis thaliana
MADHTTKEQKSFSFLAHSPSFDHSSLSYPLFDWEEDLLALQENSGSQAFPFTTTSLPLPDLEPLSEDVLNSYSSASWNETEQNRGDGASSEKKRENGTVKETTKKRKINERHREHSVRIISDITTYTTSSAPTTLSKETVSRYFYMPITQAAIALNVGLTLLKRRCRELGIRRWPHRKLMSLNTLISNVKELQKMEGEENAEKLQDALEMLEKEKRTIEDLPDLEFKDKTKRLRQACFKANHKRKKKRSLKSDQSQVPSCSSSGSVPSDESVDEAGMESDEEMKYLLCGFSSEFTSGL

SEQ ID NO: 15 - DNA, 834 bp, Arabidopsis thaliana
atggctgatcaaagacctctaatgacctggttagaggccaacaactatgaatcattccttcaagaagacatattctcgtttctcgatcaatcacttttcgtcgatcctcacagctctttcattgacccttttaaggattttcaaacccaaaattggttttctctccaagacagcattgttaatcatatatctactacctttgcggctgatcatacgtttctggcttcacttgatcttgaagctatctctagtactttctctctagatatatcgagtggatggtggaacgagaataatggtaactacaataaccaggtcgaaccaaaccttgatgaaatttcaagaactaataccatgggagatccaaatatggagcaaatattgcatgaagatgttaacacaatgaaagagaaaacaagccagaagaggataattatgaagaggcgatatagagaagatggagtcatcaataatatgtcaagggaaatgatgaagcagtacttctacatgccgataactaaagcagccaaggagcttaacattggtgtaaccctcttgaagaaaagatgtcgtgagttaggtattcctcgttggcctcaccgtaagctcacgagcctaaacgctctaattgctaatctcaaggacttgttagggaacacgaaggggagaacgcccaagagtaagctgaggaacgctttggagcttttggagatggagaagaagatgattgaggaagttcccgatttggaatttggggataagactaagaggttaagacaggcttgcttcaaggctaaatacaaacggagaaggctcttctcatcttcttcatga

SEQ ID NO: 16 - protein, 277 aa, Arabidopsis thaliana
MADQRPLMTWLEANNYESFLQEDIFSFLDQSLFVDPHSSFIDPFKDFQTQNWFSLQDSIVNHISTTFAADHTFLASLDLEAISSTFSLDISSGWWNENNGNYNNQVEPNLDEISRTNTMGDPNMEQILHEDVNTMKEKTSQKRIIMKRRYREDGVINNMSREMMKQYFYMPITKAAKELNIGVTLLKKRCRELGIPRWPHRKLTSLNALIANLKDLLGNTKGRTPKSKLRNALELLEMEKKMIEEVPDLEFGDKTKRLRQACFKAKYKRRRLFSSSS

SEQ ID NO: 17 - DNA, 771 bp, Arabidopsis thaliana
atgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattga

SEQ ID NO: 18 - protein, 256 aa, Arabidopsis thaliana
MSSSKHSSVFNYSALFLSLFLQQMDQNSLHHLDSPKIENEYEPDSLYDMLDKLPPLDSLLDMEDLKPNAGLHFQFHYNSFEDFFENIEVDNTIPSDIHLLTQEPYFSSDSSSSSPLAIQNDGLISNVKVEKVTVKKKRNLKKKRQDKLEMSEIKQFFDRPIMKAAKELNVGLTVLKKRCRELGIYRWPHRKLKSLNSLIKNLKNVGMEEEVKNLEEHRFLIEQEPDAELSDGTKKLRQACFKANYKRRKSLGDDYY

SEQ ID NO: 19 - DNA, 360 bp, Artificial sequence
  EASE promoter
ccacgatgcaaatatatcgataacgttattaaaaaaagtaaccgcatgatatattctctttcgtatgatattaaggcccacgatgcaaatatatcgataacgttattaaaaaaagtaaccgcatgatatattctctttcgtatgatattaaggcccacgatgcaaatatatcgataacgttattaaaaaaagtaaccgcatgatatattctctttcgtatgatattaaggcccacgatgcaaatatatcgataacgttattaaaaaaagtaaccgcatgatatattctctttcgtatgatattaaggcgatatccaagacccttcctctatataaggaagttcatttcatttggagagga

SEQ ID NO: 20 - DNA, 520 bp, Arabidopsis thaliana
gaatttaactgatttggtcatctttaagatcataagtattaataaggaatccaaaagttatttaaggttttgttagaaaagcaagataggcatcatgagttagtatctatatataatatagaactttttgatctttttaatcaaactatattatacatatgtcttagttcctaataaaatgtgggcttcaatagaatttttgaaatataaagttttaaacctgtaattgtttgcacttattagatgtatattactatttataccaatatataacagattttaataactaaacaattataattttttaacaaaaagcaaacgtaataggttactgaattttactttataacaaaataaaacgtttaaatgaaaattaactctttatataacatatttatctacagagcctataaatatgactaaatattgctttaatactccagagcaaaacaaaagaaaaacaattcacaataatatttaatatattttctttgtgatattggttaatttctaccaagaa

SEQ ID NO: 21 - DNA, 1419 bp, Arabidopsis thaliana
tggcagggatacccagaaaccacatttgcttacatgtcttctctataacagagtgtgtaaagttttgtgtgttgaaaggtttttaattttaagcaaaagtggattatgacgacaacagacaagcttttaattttattttaccgtaatagttatatcttgttgtaagaaaccattttcagccttttgttggaaaatcctgcttaaatggtttttgagtcttacataatagcttcttcatcttttgtcttcttaaagagaattatatttgtaatttcatgtctgttgtgtttctttgactttactgaatagagaatttgtgtgtttatggtgaaaatatagccgatctgcttgacagatgaacggagtttattttgtctggtgacatgactctgttctcttatatcaggatttttgagaaaccctttggtatctttattgtttggtctgaaggtatgtatatactttttgtctttgattaacctagtaatatgattactaactcctgtaagttcctctttcagatcactagaacaaagcaagaagttgtaatatctattgtatagtataaagatgctcgaaaaatttcagattctggttagctctagttgtacagaagaacaaaaaagtctctaaagactcaaatgtttcagaacgacctacgcctatgagtgtctaaaccggttaaatccgaaccgaaacgaatggaaacagtcttgagaaacaaaagagtaaaaaactgatcatagaatcacctagttttactaaaaagtggtatttaataaaattgctctctaaacaactttattaataacctacaacaagatttaatttctcatttcttaagaggccattaactacaagaatcacctgaaaagtattaactactcgcagccattatctccaattaattgaaaccgttttttttttggtgggaaatgtattattaatttcttaaccgttactcgcagctccaactataagtttataactatttttcgttaacaattaaaatattaattggcaccatacgttacaagttaactgattacaaatactaaggagtatataatacttaggaaaaactgtaattatatgaaatcaactagctacttcacaaaagagcaaattaactacgattggcttataaattatatccatagatcagagagatgctaagagagacgtctatccattacctaatccttaaaaaaaacgtccctcttattagcattagttaccattaatcatttatatctctctcgtaactccaaagtttttacagggcaatcaattagccgtcatacccactttcccgtacattttataacttcacttctatatctaccactacatgcatgtatatatatatacacaccgttctctctctcccgttgattagtgatcacaaacccattaata

SEQ ID NO: 22 - DNA, 579 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttattctctctccatatcttactaccactagtctcttctctgaatgtagtatataaatcttttctcgcatcatcgagtttcacaacacaacttctatctctctcactttctttaca

SEQ ID NO: 23 - DNA, 525 bp, Artificial sequence
  intein
atggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggggcaaaagcggacgaacatggcgtgaagcggatattaactatacatcaggcttcagaaattcagaccggattctttactcaagcgactggctgatttacaaaacaacggaccattatcagacctttacaaaaatcagataa

SEQ ID NO: 24 - DNA, 846 bp, Escherichia coli
atggggacaatgaagaaaaatcgcgcttttttgaagtgggcagggggcaagtatcccctgcttgatgatattaaacggcatttgcccaagggcgaatgtctggttgagccttttgtaggtgccgggtcggtgtttctcaacaccgacttttctcgttatatccttgccgatatcaatagcgacctgatcagtctctataacattgtgaagatgcgtactgatgagtacgtacaggccgcacgcgagctgtttgttcccgaaacaaattgcgccgaggtttactatcagttccgcgaagagttcaacaaaagccaggatccgttccgtcgggcggtactgtttttatatttgaaccgctacggttacaacggcctgtgtcgttacaatctgcgcggtgagtttaacgtgccgttcggccgctacaaaaaaccctatttcccggaagcagagttgtatcacttcgctgaaaaagcgcagaatgcctttttctattgtgagtcttacgccgatagcatggcgcgcgcagatgatgcatccgtcgtctattgcgatccgccttatgcaccgctgtctgcgaccgccaactttacggcgtatcacacaaacagttttacgcttgaacaacaagcgcatctggcggagatcgccgaaggtctggttgagcgccatattccagtgctgatctccaatcacgatacgatgttaacgcgtgagtggtatcagcgcgcaaaattgcatgtcgtcaaagttcgacgcagtataagcagcaacggcggcacacgtaaaaaggtggacgaactgctggctttgtacaaaccaggagtcgtttcacccgcgaaaaaataa

SEQ ID NO: 25 - DNA, 375 bp, Escherichia coli
atggggacaatgaagaaaaatcgcgcttttttgaagtgggcagggggcaagtatcccctgcttgatgatattaaacggcatttgcccaagggcgaatgtctggttgagccttttgtaggtgccgggtcggtgtttctcaacaccgacttttctcgttatatccttgccgatatcaatagcgacctgatcagtctctataacattgtgaagatgcgtactgatgagtacgtacaggccgcacgcgagctgtttgttcccgaaacaaattgcgccgaggtttactatcagttccgcgaagagttcaacaaaagccaggatccgttccgtcgggcggtactgtttttatatttgaaccgctacggttacaacggcctg

SEQ ID NO: 26 - DNA, 372 bp, Artificial Sequence
  intein
tgcctttctttcggaactgagatccttaccgttgagtacggaccacttcctattggtaagatcgtttctgaggaaattaactgctcagtgtactctgttgatccagaaggaagagtttacactcaggctatcgcacaatggcacgataggggtgaacaagaggttctcgagtacgagcttgaagatggatccgttattcgtgctacctctgaccatagattcttgactacagattatcagcttctcgctatcgaggaaatctttgctaggcaacttgatctccttactttggagaacatcaagcagacagaagaggctcttgacaaccacagacttccattccctttgctcgatgctggaaccatcaagtga

SEQ ID NO: 27 - DNA, 111 bp, Artificial sequence
  intein
atggttaaggtgattggaagacgttctcttggtgttcaaaggatcttcgatatcggattgccacaagaccacaactttcttctcgctaatggtgccatcgctgccaattgt

SEQ ID NO: 28 - DNA, 468 bp, Escherichia coli
cgttacaatctgcgcggtgagtttaacgtgccgttcggccgctacaaaaaaccctatttcccggaagcagagttgtatcacttcgctgaaaaagcgcagaatgcctttttctattgtgagtcttacgccgatagcatggcgcgcgcagatgatgcatccgtcgtctattgcgatccgccttatgcaccgctgtctgcgaccgccaactttacggcgtatcacacaaacagttttacgcttgaacaacaagcgcatctggcggagatcgccgaaggtctggttgagcgccatattccagtgctgatctccaatcacgatacgatgttaacgcgtgagtggtatcagcgcgcaaaattgcatgtcgtcaaagttcgacgcagtataagcagcaacggcggcacacgtaaaaaggtggacgaactgctggctttgtacaaaccaggagtcgtttcacccgcgaaaaaataa

SEQ ID NO: 29 - DNA, 597 bp, Corynebacterium diphtheriae
atggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctgcgatgagctaa

SEQ ID NO: 30 - DNA, 846 bp, Zea mays
tgctagtgaacctcaaggattgggggtgataaatgcgtgcttaatttttgaggatctagtaatcaagagtgagaggaggcaaaacatcgattcttcatagtgcttaaatagaaaagagtgataatactactcctttgttcgtcgagtactaaaagactactacatccattttacaattattttttagatacataaactttattattataaatctagacgtagttaagtgcaatgcaaacaacttatattttagtaatacataccattaataaataatactagtagatagtatatatatctaataagatgatattaaaggatgataataataacaattaataaatactactagtacacaaaagataagtttagcaacaattaagtttagtagtgcatgaagttgttttacgatattgataatatttatcacgcaaattttgtatattatagtgatgttttttgttccatatctatgttttatacaaattttttactgccgcaatgcactgcacatatctagttttagtactatatacaattaataaataatagataatactagcacatagtatatatctaatgaaacgatattaaaaggatggtaataatagcaattaataaatactagtagtatacaaaagataagtttagcaacaatcaaactaaaagatagccagtagaattttatttattttatattactgaaaacatcctcaagtgttcaccctgcagcccatcgcctattctatttaagaaatgcccgccctcccatactgctatcactcaagcctattctccattgtggaaccaacaaatctccaagctctcccaatttagaaacgagcc

SEQ ID NO: 31 - DNA, 1113 bp, Arabidopsis thaliana
atggtggaccaaggatttttcacactaaaaaaggaaaaaaagaaaaatatattaataaaacttttttatgttaaaatcttgggcttctgcttttgcgactcttggtcttcttcggacatggcacattccttaacctcactcgccgttttccagagcgtcatccgcaaagagatggtgaggagtttgcatgtctatgaatcggtggagattgagagagagttctggttcaagagcaaaagctgttatgtagagaagaaagcgaagcctctgtttcgttcggaagatttccggcgaccggagatctcggaagggtcggtttttggcacgtggcgttgtatctttgtgttccggtttaatcactcgcttcctcggtttcctactcttctctgtctttccagaaatcccaaactggaggacatccctaatttagccaacgagctcaagtttatctccgagttaaaaccatcaaagatttatgaagaagaacaatgcagtagcagtacagagggatattataactctgatctgcctaaaccacgaaagctcgttctgaaacaagatcttaactgccttcctgattcagaaaccgaatccgaggaatctgtaaacgaaaaaaccgaacattcggaatttgaaaacgataaaactgaacagtcggaatcagatgctaagactgagattttgaagaagaagaagaggacaccatcgagacatgttgctgaactatccttagaagagctttcaaaatactttgacctcactatcgtggaagcttctcggaatctcaaggtcggtctcactgttttgaaaaagaaatgcagagagtttgggattccacggtggcctcataggaagatcaaatctctcgactgtctcatccacgatcttcagagggaagcagagaagcagcaggaaaagaatgaagcagcagcaatggcggtagctaagaaacaggagaaactggagacagagaagagaaatatagtgaagagaccattcatggagatagggatagaaaccaaaaaattcagacaagaaaacttcaagaaaagacacagggcttctagagccaagaagaatcaagaatctcttgtcacttcctcttccacttaa

SEQ ID NO: 32 - protein, 370 aa, Arabidopsis thaliana
MVDQGFFTLKKEKKKNILIKLFYVKILGFCFCDSWSSSDMAHSLTSLAVFQSVIRKEMVRSLHVYESVEIEREFWFKSKSCYVEKKAKPLFRSEDFRRPEISEGSVFGTWRCIFVFRFNHSLPRFPTLLCLSRNPKLEDIPNLANELKFISELKPSKIYEEEQCSSSTEGYYNSDLPKPRKLVLKQDLNCLPDSETESEESVNEKTEHSEFENDKTEQSESDAKTEILKKKKRTPSRHVAELSLEELSKYFDLTIVEASRNLKVGLTVLKKKCREFGIPRWPHRKIKSLDCLIHDLQREAEKQQEKNEAAAMAVAKKQEKLETEKRNIVKRPFMEIGIETKKFRQENFKKRHRASRAKKNQESLVTSSST

SEQ ID NO: 33 - DNA, 2037 bp, Arabidopsis thaliana
atacaaaaatattttatagtagtgaactacgatatatatcattgtggactgacttgtggtgtgtgctgtctcagcgattagcaacctcacaaataaagttaatactaataagtaccctactgtttaacgacctcacaaatcaatactaataacttctaaatttgaaatttgttctctacgtttcacactacatttatggataatcgggtgtatctatagtatatgcatgcgttcgtatgagttttaataccagcgttgactgtcggcaagtaggaaataatccaattaataatacgtttgacaaaagattaaactgtagtactatatataatggaatatttaatccagatatcaaccgttgaaagttatctaatttaatttgataacgatttccaggactgtccccaaatctatctgaaagttattaatcactcctttctaaacaataattgaactttttcttaaaaaaacttctacgacaacacatttcctttgcataacgtagaagtcaatcaaagtttttaaatacttctatcaaatttttaagtaaaatagtattgacacgaaatgcaaaagacgaagtatactgaatataaaatatcacggctacaatgcaacatttaagaattagatgattggaaatcgatacagaaaaataatctaagagaattaggccgtcacttgtgttgtgtgggagcaaaacaaggaccaaaaatatcgggacaaataggttggtccaacctataggtagaggtagcccacttggcatagctcataataccattaccagctcatatgttttttcaaggattggagaaaattaaagaaagatgtaatcgattagagtaacagtggagtgctgaatttaagttagttaagaaaataattggtgttacttcttataaacttttaactcaaaaccaattcgtaatgaatagatagatccatgtctattatatcttatatactattcaaacctcttcttatatatttttccaatgtggattattcgcccatagataaaagataaaacttaacaattggtaagacaatatgacataaagtccttagttctacttacaaagaattttgtcaattaccttccaaaatttagatcttctaaaccctaagttattgggtttcaccaatataatgggtcatttcatctattcacccgaccgttagatttaccaatttctcatcatatctcgattttcaacatttaagaaagtaatcaagtttagccgaaatgcaagatgatacagaaacaatagcgtttaacggtgttagatgataaactcatcaactccattaagaaaaccaatcctgtaagaggtaaagaaggggagaccataattaatgtctaatactttcgtaatgaccactattaatgattagtactatgatctatgaagttgaagctctctttttttttttttttttttcccttcacgtccatagttagttacagcattgatgaaatttttgctgagaatagacgaccctttatcctccaccctacgctttaagtggttgggagttagaccctgccagatagattccaatcctaagataagtctgtttaacaaacctatcatatgtgaaagtgaaaaccattatgttgaagaattatctaaggcgtagagataatttctgcagcaaaaacatttttttaaacattgcgttatacattttaggatagtttatataatcagccaaagtgtatatttctgtaaaacacattactatcttgacatttttgtgataagctatataatcagtaacctgctacgtatagcttaaccccactattataattatgattcctcattcagtaaaactatatagctgaattaataaagtttattagggtctaatgaagttggtgtgatcatttaataatattgttatttcataactcggaattgaattatttattacccttgccatcttaaatctacatttgcaactcacccaaaagctttatcctttgtgttttttccactgtatactgaaaacaaatctgaggtgacgaag

SEQ ID NO: 34 - DNA, 1358 bp, Zea mays
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccatggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggactcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgaatgcacgcactaccatcaattcgccgccgcggccatcccttctgccagctgctatttatacgcctcgccccgctccagtctcagcagaaccaccagtcctccactccatcttctactccgaccacaaccacagcgaccacgaccgtgcacgtacgtacatgagcacaccaggcaacggcacc

SEQ ID NO: 35 - DNA, 30 bp, Zea mays
acacaggaccaagaacttgaagatgcattt

SEQ ID NO: 36 - DNA, 25 bp, Zea mays
gtgcctagcttattcgacgacctcg

SEQ ID NO: 37 - DNA, 25 bp, Zoanthus sp.
agtccaagcacggcctgaccaagga

SEQ ID NO: 38 - DNA, 25 bp, Zoanthus sp.
tacacggtgtcgaactggcagcgca

SEQ ID NO: 39 - DNA, 26 bp, Anemonia majano
atggccctgtccaacaagttcatcgg

SEQ ID NO: 40 - DNA, 29 bp, Anemonia majano
ggaggtgtggaactggcatctgtagttgc

SEQ ID NO: 41 - DNA, 1262 bp, Arabidopsis thaliana
gtttaggggtaatttagtttttaaaatatcatttatgtgttcttggaagtaacatattaatatcttaacatgaaaatctttggtcttggggttttggttttgcaaacttaattctctgatgttgaaatttgaccatctcttataatatttagaagtttgtgctttttgatagtccggaggagtatgaatgatcaatgaaccctttcaactgtgaaaatttcgagtagattaatattaataagagtaaaattttcattaaagaaaattttcactaaagaaacaaacaaaatatcaaattaactaaattaataaagccctcttttatcagaaaaggtggcctacttcaaatgttagggtgtcttattggtttgtgatttaaataaagtttttgtaacttaaagtgttatgtaaaatctgttgttattcaatcatttttatacaaagattttgatgtagtttagtgttatttgtttaagattttgtaaaaagtaatttaaaatcttcataaatctagaattattggattcatacttttataaaattaataaagttttgtgttgttaaattaaaacaaaaaatctataattgttaataaattaaattattatgttattagtttataactttctacactttattcataaaataaagttataaaaaatatcatcaaaataagagattgtttggaaaacttacaaaaatattaaaaaaaccaatcaacaaaattataaaaaataagtctctaataattatttaaaatctatttactttctataattttataaacgtcatcaaaattatcctcgtattagttttatctggtgactttgggcattttccctttctcataaaagggcgcgtgactcaaaattaatgtatagatgtcccataatttcattaagaatagattgttattttaaagtaacgtatcttttatttatgtagacaatattgttttcacgcatgtcttactaatgatgataatatataattaataatgaagacatttattaggtcttatcaattatcaggaaaaaaaagaaagacatttattaggtcaatttgctgacgctataaaagaaagaccttatcatttgattccaacacaattcatacaaacatcttccaagtaagtgatttggttttgatcaatctttaacaattttctcgtattacaacaccatcaaactaacaagtaacaacaatcattttttctattttatttgatgaaaagggaaatagtttggtgatttctcgtaaag

SEQ ID NO: 42 - DNA, 1834 bp, Arabidopsis thaliana
gctttaaagtcgtttatttttgtaacattactctctatttttgaaaaatgcgaaataatttttcaaagtaaaaaataatatgcaatttaggctttatacatatattataaacgttttttcgttcacatacatttgattttcaaaaatagaaaggtaagttgaacttttcgtctcgagttctttgaattgatatattacttatcaaattttaaaaaatatgagaaaacttaacaatagcaatattatgtattattttttactttataaaattattctgcaaatattgtgaattattttttacttcaaaaaattattttgtattcttttaagatgaaggataaagttataaaaatagacgactacaagaatttttttccacaaatctcctttttattcagatggtcaaacatggtcaaattgatacataatccacagaagttgtagagagattatagatgatggactctttgtatgtcattctgttttttcagacagctaaacgttatttaaaaaataaaaatacaatgcattaaaaacaaccatcctcgacttgtgctcacgcaacgctaccgtcttcatcattttaacctctctcgaccattttaacctctctcgaccctttttgtttttcattttttttaattaattattttcaaactaaccgaacccaatcaactaaatttacccctatttaactcaattttgaccagaaaaccaaaaagttcgattaatttcgataacaaaataaaataataacatggttcttaaacccaacccacacgaagaatcggactgccttttggggccacttggccattgtgtcaaccgggtttgaccacaagtcaattaaaaaaaaattatttaatatatttaatatttagaaaagttatatagtttatattaaataaaaataaaaatagtaataccaagtttaacaaaagtctaacaataataaacaactaaattttaattaaatttgatgaatactaaatcattgtaatattcgatcgtcattttagtctaacaataataatcaattaaaattttatttattatttttaagtccaactaaaatctaaaaccataacagaaatactagagatcattgatgacgaaaataaactaagaaaacatcacgaatttaaaataatgaattttgttttttctctctcacaattctattcattctttaaaagcgggattgtgaagtcttcaccaaatctaaaacattaaatgatgaaaaagttctaaaaataagtgaatatagtttgaaaccctagattctattccaaaatcaaatgaaaattttaaaacccatagccggcctgttttaatcgcttcaccagatcgcaagttaatgaagggtttttttgtggatttttctggttttagattgtcgagtattagttctaaacccaaataggaaaaatgtccgggtagcggattaccatgtcggaccggacggtccggatcaggcgtgaaaacaatgcatgtaatcgtattgtgtctaatatagtatttttgatttgtaataatttgaagaaaaaagagagtgttgttatctttaagtttgcccaaaatctacagtaatgttcgatcatagtctttaaagagagtgttgttatctttaaagttacaactttgtaaaattagcatagtctttaatataaacgtatcttaaacaaaattattaaatgttgaagttagtaacatataactattaattaatgaacaaatatcttttagtgattaacctataaaatctcttgttttcttgtttcatgtcatcaatcttacattcaatactaaaagtattcttacatccataaaaaaa

SEQ ID NO: 43 - DNA, 1248 bp, Arabidopsis thaliana
ttagtcagcaaaatcaaaatttaacatttaaataaagtctttatttaatattttatagcatttataatttgaaaatatgtaatgcaatgataaaaaataaaaataaaattctattatatactgaaatgatatccaactttttatacattccaaaactatatttggatgtctcttgatctcaactctgctcgtaggctatctaacaagtcagcagcaatataggtcttcagtgggccttattgggcctcattatgataagtaaagttctcgtagtggcctacaaaaattatattgaggggaccagataatagcttcacgtttagaagtttcataaagggaaaactcatatttcatttttgttattgttgacgtataaacaatccagatcatgaaaaaaaaaaagcgtataaacaatcttaaaattctaaccacttccaaattagtttttctcgaaactatttgtgcttttttgtttgttttgcttttgtggattttgattggagaagagaagaagaaatattatatgttttgcgtttgcatttaggttttttgtttgggtttagaaatattgaaactgatgtcttaactcttaaaatatatatttagcgctattgtctaacgttgatgtagtttggcatttactttttttaggtatgttgtatgcattagagttaattgtttgcttttgcattttcacatttaatttgaatgtgtttgcgttcaagataattaacattatttgtttgtgtgttttctttgaaattaagaagataatttgagctaccactgaattttgaaattagagaggcatcgagggaaacaaatcatatagtttggtgactgatttcaaggggaaataaccaaagaaggtcattagaagaataaatatggttagccagtattgattaggaagataatcaacatgttgaccacaatgaaagttagtcaatgaacggttttcaaataaagattacaaaataactagaccataaaaggtgatattctataaattctaattgttctttttatgtgttgtaataataattgttttattttaataactatatgtaaaaattattgtttatttatttcttatatattatggatgtcacgtgtataattatgaaaatccacgacttagaatgttcatgcattgcaattgtaagaaagcacttatgccttctatatatatattcgttgaaatgaaaacgataagagcacaaaaacaaaaacaaagtagaaaaggat

SEQ ID NO: 44 - DNA, 3674 bp, Sorghum bicolor
cggaccgaagctttcatgaatacggccttgctcctagggttgagcactatgctgcgcttgtcaatctcatagggcgacatggccagcttgaggatgcactggaggtgatcaagagcatgccaattgctccagaccgagctgtgtggggcgcattccttggagcctgcactgctaaaaagaatgaagtgctggctgcagtggctgccaatgcattatccaagattgatcctgagagttcagctccatatgttttgatgcataacttacatgcccatgaggggaggtggggaagtgcatctgtggttagagaagacatggaacggctagggattcacaagcatccagggtacagctggattgatctgcacgacaaggtgcatgtcttcatctcaggggatacctcgcatccccttacccaggagattttttcagtgctagaatgtttttataggtcatgtagagattggagctagacggccatgtgaaattgttatatttggagaagagaagaggttttgcggtgtagaaacaagctctttcttccgtttcttcttggcctatacatgtctcttgtaatgtttgtacctttctttggtaatgaaaacacaataattttattattacatttgataaaattgaagatccatctggttgggaaggctagggggatttgaaggactagttttcccaaacaataacccggcgacagtaggggtcatacgatgtcaattctaaccctctggtgcctatggatccaaagaaacggagtggtttttagagggcaggagaggtcaccattagacgtcctgagggacaacaaagacacagcatgctgctgggctttagctcgaccccagacggctgctccacctgcaattggttccctaggtagtgagtaatctcttttctgttttcatgccctagggcagcctagactgttttcaggggagcgctcctcgtgcgtgtatgctactattcagcttcctccttactattaatcaaagccggagttttccggatctttaaaaaaaagagagagataaaattgaagatctatgatggcactgctgattgtgtgaaaactaaagtactctcatacagatttccataatagtgatgtggctgtcaaatatttgcctgcaacttgaagaatttaaaatggttgaaattacatggagatgagccaactcaactgctcaagtaatctctcaccccctgccacttgaatggatacataattgccttttgcctatgcatgataattattgctgtaatgatcagttcataaatttatgactaaagtaaaaaccttagccttaacccaaatctatgatattagctcaggcaaagagtatatgctagaaatttctatcattttaattgagtagcactaatcctttgaaatgtgtaaaagaaaagttctagtatgatattagctcaggcaacccattgagtcacaactccgtgctacttctacttcccaatgaaaaaaatgccatgcatagatggcaaagactagcagtgctcctagattccttcgtgcaagtagaaacaaaatcttgaactgaatctagccggaaagactttgattgaccactatgcatgctctctaatgcacgaaccccaatggcatgctcggcaattaccaagagctaattatatctgtaactcccgatccattagccaccctttgcattaattcctcgcgtggtttttaatggccgtttccattaacccaatgatcccagggtttaaaagagccgcatttttccttccatcttgatcttctccatatattgctggcctcaactccgttccagcatctcctcccggaacccggaccgaagctttcatgaatacggccttgctcctagggttgagcactatgctgcgcttgtcaatctcatagggcgacatggccagcttgaggatgcactggaggtgatcaagagcatgccaattgctccagaccgagctgtgtggggcgcattccttggagcctgcactgctaaaaagaatgaagtgctggctgcagtggctgccaatgcattatccaagattgatcctgagagttcagctccatatgttttgatgcataacttacatgcccatgaggggaggtggggaagtgcatctgtggttagagaagacatggaacggctagggattcacaagcatccagggtacagctggattgatctgcacgacaaggtgcatgtcttcatctcaggggatacctcgcatccccttacccaggagattttttcagtgctagaatgtttttataggtcatgtagagattggagctagacggccatgtgaaattgttatatttggagaagagaagaggttttgcggtgtagaaacaagctctttcttccgtttcttcttggcctatacatgtctcttgtaatgtttgtacctttctttggtaatgaaaacacaataattttattattacatttgataaaattgaagatccatctggttgggaaggctagggggatttgaaggactagttttcccaaacaataacccggcgacagtaggggtcatacgatgtcaattctaaccctctggtgcctatggatccaaagaaacggagtggtttttagagggcaggagaggtcaccattagacgtcctgagggacaacaaagacacagcatgctgctgggctttagctcgaccccagacggctgctccacctgcaattggttccctaggtagtgagtaatctcttttctgttttcatgccctagggcagcctagactgttttcaggggagcgctcctcgtgcgtgtatgctactattcagcttcctccttactattaatcaaagccggagttttccggatctttaaaaaaaagagagagataaaattgaagatctatgatggcactgctgattgtgtgaaaactaaagtactctcatacagatttccataatagtgatgtggctgtcaaatatttgcctgcaacttgaagaatttaaaatggttgaaattacatggagatgagccaactcaactgctcaagtaatctctcaccccctgccacttgaatggatacataattgccttttgcctatgcatgataattattgctgtaatgatcagttcataaatttatgactaaagtaaaaaccttagccttaacccaaatctatgatattagctcaggcaaagagtatatgctagaaatttctatcattttaattgagtagcactaatcctttgaaatgtgtaaaagaaaagttctagtatgatattagctcaggcaacccattgagtcacaactccgtgctacttctacttcccaatgaaaaaaatgccatgcatagatggcaaagactagcagtgctcctagattccttcgtgcaagtagaaacaaaatcttgaactgaatctagccggaaagactttgattgaccactatgcatgctctctaatgcacgaaccccaatggcatgctcggcaattaccaagagctaattatatctgtaactcccgatccattagccaccctttgcattaattcctcgcgtggtttttaatggccgtttccattaacccaatgatcccagggtttaaaagagccgcatttttccttccatcttgatcttctccatatattgctggcctcaactccgttccagcatctcctcccggaacc

SEQ ID NO: 45 - DNA, 1808 bp, Oryza sativa
tttccatcctatcgagatgtactactccacttctgttctgtgcaggttgaatatatgtggcccaatcacatcttgccactaaaaatcttacatttatccatatactccacgaacagtagattttactcatccctgattagacccaaaacaatcatgagcacggtagacaacacaagcttagggcgtcttgcacgattaggttttgttcggtttagaggggattgaagaggattagaggggactgaggggtaataatttcacaccataataggtattgaataaatcccctctaatcccttcctcatgagaattaaccgaacaagcccttaccccgctacacccaaaaatgtttccgctggggtgcaatactgctatcgatggcttcttacgtaggaatttcatttttctaatattttttcattaaaaattgtacaaatatgacaaatctcttttataaaacaaaggtttctatagaaattatgcgagcacatatgttcacatatacacatatttcatatttatgactaattatttttttcaacgacaccgacaaatccgtcaataggctttatttttctttcacaaagcccgtaaacttccataggagcctactacatcagtggcttcgtgccgcactaacgaggcatctatagtgattgactttatcaatgtaaaatatgacagccaaatattttgatgggaggtgttcatggttatatgtacgtttatactccgtatgagtgagtagcactccctccgttctgagatatttactagtactacgaatctggaaatactctttattcagattcattgtactataaaagtatctcatatatccaaaaatttttatattttgagaccgagtgaatatatgtttgtggttttcctacatgtgagtagagtgcatcagtggatattagagcctccacgatatgggaatagtatcagccagtgtgttgatgacgtcaaagctcaaagggtagatgaaaagttcatgcttcaaaaatggcatgtcttggaaactgggattttcctaataatgagaaatcctatgtgcagagaggagacaaaagcactgctcaacacactgcaggctgcaaagatttgctagtactactactccagtacacaaacacatcattggccacttccctaatctcatttaacgtttgcataacgcactcattctgcggttactgcattagctactcatgaatgtggctatttactagtagtacaattctaagtgccattcccaggaggagtgagcagcttctccacccttaatcaggggcggagctaattggttttggcgatcaatctgcctcgtcgagtcgtcgttccgccctccacacttcccagttcgcgactgcgccaacgattgcgcgagcaccgctgccgcaactcaactcccgtgaccgacggcggcaatcggtggccggcgaggcagcgatcaggatcagggtaagtatatttcatctcctcctcctgtcctttggccctcccttctctgatccctcccgtcttcattaagctctaatcctaggtactaaattactaatttgattagtaagcggttaggccactagaacttgcgcccttgccgacggccaacacgacgctcgcaggccacaagacaaaagctgaatgaagcaccggcatcgcatgaactgatcgcattgtgttggtaaattctatacttctatgtcgacatattacatttatagtgttaaagaaaatttatgttcagttggaccatcctagcctaaaatcgtagctacgccactgcccttaagcccttgccc

SEQ ID NO: 46 - DNA, 582 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttattctctctccatatcttactaccactagactctatcagtgatagagtatataaatcactctatcagtgatagagtttcacaacacaactactctatcagtgatagagtttacaatg

SEQ ID NO: 47 - DNA, 582 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttattctctctccatatcttactaccactagactctatcagtgatagagtatataaactctatcagtgatagagtagtttcacaacactctatcagtgatagagtctttctttacaatg

SEQ ID NO: 48 - DNA, 582 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttactctatcagtgatagagtctaccactagtctcttctctgaatgtagtatataaatcactctatcagtgatagagtttcacaacacaactactctatcagtgatagagtttacaatg

SEQ ID NO: 49 - DNA, 273 bp, Bacillus amyloliquefaciens
atgaaaaaagcagtcattaacggggaacaaatcagaagtatcagcgacctccaccagacattgaaaaaggagcttgcccttccggaatactacggtgaaaacctggacgctttatgggattgtctgaccggatgggtggagtacccgctcgttttggaatggaggcagtttgaacaaagcaagcagctgactgaaaatggcgccgagagtgtgcttcaggttttccgtgaagcgaaagcggaaggctgcgacatcaccatcatactttcttaa

SEQ ID NO: 50 - DNA, 1314 bp, Arabidopsis thaliana
ctgagaaggacatggtcggtgatcatacacggcgaggtggaaatgttatatttactattgaaaactaaattatttattatagagggagatattactctttacgctttcattaagatttatttttataagttttaaagtattttattgttatatgaagataaaatatattatttatttatattttattttataataagatattattttttatttttttttattattttatttttattctctgtgctatatatactctgaaagtctgaatatataatccattttggtgtgggagtattagactattaattatggtcaattaaatgaagttcaaaaatatgaatggaagatatatgaataaattgaattaatagatgtttataattattgagactgctttagcgtagaaaatgctgcatacattattgttgggaaaataaaaatgagtattaatatttaacataaatattaaatgtctttaatatgtgtgagagaattattaaaaaaaatcaacatttacgaaagagatggactataaacatttcgttaatacattttgttttttggtaaattggtttaatacaatatttttgaatcgtaaagtgttctggtaatatgatatgacatctaaatgaaatgattatgccagaagatcattgtcttgaatattggctgtattaacctctaacgaaattgagttaatatatattttgaatttaccatttgatatttagattgtataatttgagtttaccagctatatatcgtgttgaacttgcatgtaacacaccacttttttccaccgatttttgtttatggaaatataagtcaatatttattcgtcaaatacatatatactcacgcaaatatacgtccttaaagagaaaagagattttcatgattatttttgaaaaaagagaagattttgaaagatgacaacaagcaacgatatatgaacgcgcatagcatgtgatgggatggggcgggcctatgaaatttttgaacgtttacaaacttagggcctattattagaagatattactagcttttaataaacgaattatccctattaaccaaaataatcaacactaatcattaatttctacttactatctctctcgtaacttacagaaaacatataatgattttgacggctcatcatctcggagaactaaatacccacttcccacttatcatgtactttctctatctatgcatgtacgttaagttgtttatatatatatatacacacgattcattttccttgttttaagactaacgaacgttacaatctatctatatccactttcaatcgaa

SEQ ID NO: 51 - DNA, 654 bp, Arabidopsis thaliana
aacaccaatatgaagagaaaaaagcttgattctttctcattactcttcaagaactcaaaattacattgtgttttggtgtttcttcttcgagctcaaatcatcttggggttttcacagatttattcaaacaatgtactcccaagattattattgggagtattattatgtagtgcgaactcgatttgagaagtgaaaaaaagatggttacatttaaagcttttgatttgactacgttttctttgtttcatttactaagtaaattatcacttagtggagactctcattatctcttaatcatcttcaacatcaaatgtatctatcatcgtaacatataacacgtgcatcatctaatgcgataatacacaaaaactcaattcatttaatatcgattgtgaatttttagcaatatgatcttatcaactttcatgcattgactttgactagaggaagtagaaaaaaataatcgtcatcatcattaaagaagcaactaacctacacacaattcagcccccgtgatcatatatacttaattaaagtcacacggtaattaattaagattaacatttaatgatttctaatacgctttgggactcgtaactcccattacattgcaatccctatgaacattcatctttgtttttacagagactatat

SEQ ID NO: 52 - DNA, 2164 bp, Arabidopsis thaliana
aatcctcttctttagggtttctttccgactttgaatacactctctgcttttttttctgctttctaaaaagtcttcaacactttgctttctctcatcttcttttttttttccctctttttttttaacctttctttagaccacgtgagaaagataacttccactttaaacacttgtcctcttctgtcttattgtcttgtcttgtcttttcttgataggcttccattattgtggctagggcccaaaaaggccttaaagcccaaagcttcgtggtttttcttctcttgtggtttaggctttacagtgatcagagaaacccaaaacacgttggaaacgtctaagcagagaaaaacagagcttccaacaaattcagcattgtaattcttctagacgttttatacaaattttacatatacactatggaactctccttgcatttctaccaaatctgaattgaaaaagggatttgtaagatatgaaaatgcgataacgttgcctagattaatcagttttcgacattttttttttcctgttccgattccatgtaactttttgagggccacaacttttcttaattaaaaaaataagaaaaataaaagctcaagtgacaatcagtttttgaaaatgatactaactaagctcttaacatttttacgcatgtatataaacattaatcttttatttggtcttaaatacaaagcatatatatgatgctatcaatctaaatggtctatttgtacataattaaataaaacataaaattaaagcctgcgcatacaacatgtctacaaccaaaaacttctttcgtttatatcaaaatcaacatcccaatacttcatcttctcttctcttctatttggcacttatagacgcgaaaggtttgaaccggcgggaaagtaagacaccataatcggagctctcggggatttgctttttggtttctttgaggacaggactttcaagtcactctcatcagttgagctaatctttgagtctgattttggaacaaagcaatcaaagacggaggcaaagagagaacacatgatcataggagtttgaaaaacgtgtttggagtctatatacgatgaatgatatgattaagatttgatctcaggtaattacgtgaacgatatgtatttataagacaacccatctttataaattcttggacacgtttctaggaaatgaccactaaatcttgctggccaagctttgccctattcttaattgttttctcttttgacaacacttgggcaccttttttgactctttgggcctaattggaccaactattgataccaaacatacgttaacatcacctccatatcactcacccaatcaagttttccaaaatgttatgattaaaattagggtcttcatgtcactatccaacaaaagttttccaaaattcaacattaaaattaggggaaatatgtacgaaatagaacttatatatccatgttaagaagaaaaaaaactatatatccaagcaatacaaaatatttaggttctacactccattttatacaaaatattaattgttttcgattagagttttattagaaagttctcactcagataaaatcaaaactagtactctgtatttttatatagagaaaaatccttgtaagttaatgttactaatactacccaagtacccagagtattttgacacattctattgacttttgattgaaacatgtccggcttaatttaacgcaattattcagtttagattttgaacaccttaaatgaattggcttttaacagatcataatatcaataccagttttagtccttgagaactcagcccatgacttaaaatatgaaaacttcagcccatgacttaataaatgaacaaagagaacccaaaaacagaaaatgaatcatggacatatttacatatatcataatctgaccaaattggaaattatgctcaaatgcttaatattcctctgattcatttaccaaattcaacctctgtagaatcattctaaacaaaattcaattaccacttttcagacatgcgtcgcgcgtgtgagtgtttagctacatgggcttggttcggtgcaacccgcttcccactgttaattttacataactaccctcgcacgctccgcttgcctacacgtgcgttccggaatattctgcctttttggtaatttcg

SEQ ID NO: 53 - DNA, 978 bp, Arabidopsis thaliana
gtacagggaaaaatgcggtgtaaataccaaactttacgaagcgtggcaaaaatgttataaaaaaaaaatctataaaactttgttattgtgatgtgaaggaatcgccctagtcaacaaattaaatcacaatcacctcatgaacacaactgatttaactatatcaactttttcttgaaccaaaggtaccaagtacaaactaataacgatacgagttgtcagttgtgtaccaagtttttactggcaaataaatcgacttgctaccaaagtaccaactaatacgagtgtctctgttttgttaacttgacccaatctttcttcctcgtctctttgcaaaacgcttaagcccaaatataactaatatggcccaaaatattcttgagagatccaaacctataactcgaatacccggtaggacaaaacgcttcatgtcatattctgacactttttaacacttcatgatcggtatttaaatagcattttcatttcttgtataacaactgagttcatatatatacatcattgatcatatattgagtattgatctaactaattcataatcaactattcaactgttttcattaaaaaaaacaagtttcgtatataaaacttggaaatattgtttttaattaatttgaacgtacattgttatgggttcttctaatgttaagaaaacaccaaagagagaaaaaagggtggtcaaaaaacaaatttagaaatcaatgctataattaagctatgataaactaatcatttttttatcgaaacgtaatgaaactaattttaaattttaacaatcaacgattttacttttttgtctcagtctaaaaataacaatcgggtttctaatataaaacaaactcggtgctccacgagaatagttgtcctcttctcaaacatatctcaacttattgtttgaatataaaaagagatatcaaaaagaagagaagaccaaaaacaaaacaaaaatctctaataacc

SEQ ID NO: 54 - DNA, 524 bp, Arabidopsis thaliana
gaaaattgtgcaaaagctttcatgtgcggctcagattaattagtcatttactactaataaaactttcactttggggtctagtagataattctccacccccattgaatctttttagtggaggtctaaacatacataagattctatagattgacatttgggaaaccatcctcatacaaaaaagacctaaacggaatctatgaagaattattaacagaaaagaaaaacagatggcaatgagaaaagatcgggttcaaggaaaacacagccgtacaaaactcaagaacaaaaacaccaaaaataaacaaaaaaacttccaaaaatagatatagaatcacatggttttgttgttttgtctatttgttctctataaaaggagatatttggttggatctatcatagcgtctcctctcaaccaaagcttacaatttgttctcccttaaaaactaaattttacaaataaactctcaaatccaagagaggagaagaccgaagtaaaaacacaagaaaaaaaaggattaaggcac

SEQ ID NO: 55 - DNA, 995 bp, Arabidopsis thaliana
atttagaagtgggaatgggtctatgaaatgagattacgtcaatatgagtgaaattgataaattatccaatcccataaacgagatggtgaacaaatataaatttacatttactgctagtaaatacaactacaattactttttaccacgcaaaaggagagaggagagattttttttttttttttacttcgtaaggataatatgtacttagaaaataatatacagtgacgaaggatgatgaatgctttcatgggaaacgagcaattgaccaggttgagagagatatgggccgattaaagctgtcactgtctctgttatgacagaactaagttcacgtttacgtgatttaaatttttattgatagaggagatgattgtgtttacaatcactgaattgttactgattttactgtgaattgcatatcaattggtaaacctgtaaaattgtcttatcattttgtggattaccaatcatatttatgagaaatctcaattccatttacataaatatttaaaagacaattacagaataatttagctatgacgctccgacataatcaacaaacaaaacaatattttgcatctgtatatatatatatacaaaattttgttacacatacacataattttgaggaagaaacaaaaattattatttggttgcaattttagactgttttataattaaccgagtaatattgatcattctcaaccacttaatcaattgattctttttttttttttttttgcttgatataaaaaaagttacggtaaaattggaaatcgttactacctaagattggggtcaacaatccgtaaaagaagatggaatcacacactgtaataccaatacttttctataaggaatcaaatctataaatagcatactaactagcactataaaaacattatgaatcctcctatgagcaaatcacttttaaatttgttaacactcttttaaaagaacaaaaaaagcaaaaaaaaaataaagatattatcacc

SEQ ID NO: 56 - DNA, 1783 bp, Arabidopsis thaliana
actcaaaaggcatagctacattaattctcagaaaatcatcaaacaaatactttatgttataatcactagctagtaaatgtttttttttttttgtaaaataaaatcaagattggtatagggcaaccacagatctattgatcgacctatgctaggataactctgtaaaaacaaatatagattgtaacaaacattcagaagtgaggcgagctcacattaataaaagtttttgataattttcgtctcaacacaaaagtaattaagcagttataatcttttaccatatttcataattatgatcgctacattaaaaaaaaaatctacttcaatttcatttttcatttttatctttgcaatgacctaacacaaattcttccatgagatcaaccttttcataagaaagggagattgaatcaaagaccaccataataaattaaaaatactgtccaagaaaaaaatagttttgttgacgccaatgatcgaatatgttataggattgtgcttttttctatttttgcgggtaattgtgaggttacttcatgaaagaagatcaacaatctttgcggccaatttggtaagctacaaaactaagcctatgtctgagcagttcacgtaagcttctctagtggctcttcaatccaattttcaaactaaacgtgtgatttccacacttaaatctcacgtatatttattcggttcttatggttccgagacaggttctggtctagtgtaactgagaaaagctccttataaatttctgcatgtttctatttttaaccgtttgcatgcaattcatacaagtttagtaagggtttttttttggggtcaaagatgccagttttagtagttcttaaaccgattttgtaaaagctatggacgattcgaatttatctcctcggaagattgtatataaaccataatttatacgaatgattgatttttggtagtttaattggtctttgtgagtgttcttagacttttctcttgatggttgtttgatcttaaaacatttcccatgtgaagtctaactctcttatagtattatacaatagcaaaaacatgttagagattttaagagaattgaatagtttaattattttagtcaacttattttagtttaaaccttttaacatttccaccatcatacaaataaactatttaattaacactttgtaaggtgtaacactttttagcatgtatgcattatatattattttgtttaactcagtgaagtattcatctgaatacaagttaactatgaatatatagtcctgtcttcttacatgaaagagtcatattttaataccacatagcaacagcaataatattgttacatgctataatatcagagcatccacaaagacaattggtccactagtcagagatgtacctagcttatgttgagcgacaagaaatcaaatattttggtacgtacagtgatcaacatgtgaatagtaagatatgcaacccgatatacagtcatttacataactagattgatgatccataaagaccgaaaaagtagtggtcataaacgaatgttgcacaaattttgtttaagagtcagttacataataatttgcatctaaatatagattaaagaaaaatgcggatcacagcaatagaaattgccgtcaaaatagagagtgaaacaagagaacctcttttgctattcaattgcaaccttaaaccaatccaccattttctcttattcacataaaaaatagagttttaaccatctatataaaccccacctcacctagaaagtaaaatcatcccaaaagga

(19) United States
(12) Patent Application Publication
(10) Pub. No.: US 2015/0152430 A1
(43) Pub. Date: Jun. 4, 2015

(54) COMPOSITIONS AND METHODS FOR THE EXPRESSION OF A SEQUENCE IN A REPRODUCTIVE TISSUE OF A PLANT

(71) Applicant: Pioneer Hi-Bred International, Inc., Johnston, IA (US)

(72) Inventors: Marc C. Albertsen, Grimes, IA (US); Mark Alan Chamberlin, Windsor Heights, IA (US); Tim Wayne Fox, Des Moines, IA (US); Shai Joshua Lawit, Urbandale, IA (US); Brian Roy Loveland, Collins, IA (US)

(21) Appl. No.: 14/612,420

(22) Filed: Feb. 3, 2015

Related U.S. Application Data
(63) Continuation of application No. 13/445,440, filed on Apr. 12, 2012.
(60) Provisional application No. 61/583,648, filed on Jan. 6, 2012.

(51) Int. Cl. C12N 15/82; C07K 14/415

(52) U.S. Cl. CPC C12N 15/8233; C12N 15/8234; C07K 14/415; C12N 15/8263; C12N 15/829

(57) ABSTRACT

Compositions and methods for regulating expression of heterologous nucleotide sequences in a plant are provided. Compositions include promoter sequences with direct expression in an egg cell or embryonic cell-preferred manner. Such compositions find use in, for example, a method for expressing a heterologous nucleotide sequence in a plant; detection of specific cell types in the ovule and targeted ablation of specific cell types.

### CROSS-REFERENCE

This utility application is a continuation of co-pending U.S. Non Provisional application Ser. No. 13/445,440 filed Apr. 12, 2012, and claims the benefit U.S. Provisional Application No. 61/583,648, filed Jan. 6, 2012, each of which is incorporated herein by reference.

### FIELD OF THE DISCLOSURE

The present disclosure relates to the field of plant molecular biology, more particularly to regulation of gene expression in plants.

### BACKGROUND OF THE DISCLOSURE

Expression of heterologous DNA sequences in a plant host is dependent upon the presence of operably linked regulatory elements that are functional within the plant host. Choice of the promoter sequence will determine when and where within the organism the heterologous DNA sequence is expressed. Where expression in specific tissues or organs is desired, tissue-preferred promoters may be used. Where gene expression in response to a stimulus is desired, inducible promoters are the regulatory element of choice. In contrast, where continuous expression is desired throughout the cells of a plant, constitutive promoters are utilized. Additional regulatory sequences upstream and/or downstream from the core promoter sequence may be included in the expression constructs of transformation vectors to bring about varying levels of expression of heterologous nucleotide sequences in a transgenic plant.

Frequently it is desirable to express a DNA sequence in particular tissues or organs of a plant. For example, increased resistance of a plant to infection by soil- and air-borne pathogens might be accomplished by genetic manipulation of the plant's genome to comprise a tissue-preferred promoter operably linked to a heterologous pathogen-resistance gene such that pathogen-resistance proteins are produced in the desired plant tissue. Alternatively, it might be desirable to inhibit expression of a native DNA sequence within a plant's tissues to achieve a desired phenotype. In this case, such inhibition might be accomplished with transformation of the plant to comprise a tissue-preferred promoter operably linked to an antisense nucleotide sequence, such that expression of the antisense sequence produces an RNA transcript that interferes with translation of the mRNA of the native DNA sequence.

Additionally, it may be desirable to express a DNA sequence in plant tissues that are in a particular growth or developmental phase such as, for example, cell division or elongation. Such a DNA sequence may be used to promote or inhibit plant growth processes, thereby affecting the growth rate or architecture of the plant. Isolation and characterization of cell type-preferred promoters, particularly promoters that can serve as regulatory elements for expression of isolated nucleotide sequences of interest in egg cells and embryonic cells, are needed for impacting various traits in plants and for use with scorable markers. In certain circumstances, ablation of specific cell types can result in damage to target cells without harming surrounding cell types. Preferential cell ablation could be used to produce female sterile plants for applications in apomixis or the production of self-reproducing plants. However, cell type-preferred promoters are needed to express cytotoxins in a spatially and temporally controlled manner.

It is often useful or necessary to monitor the induction, presence, development or ablation of cells of a particular type, for example at a specific point in time and/or under specific conditions. Cytological or genetic means are available but have known limitations. For example, great skill is required to identify the different cell types within an ovule. Simultaneous use of multiple fluorescent tags within cell types associated with the ovule can facilitate identification of the presence, growth and/or ablation of cell types therein. Other examples provide for differential labeling of cell types to track cell development and cell fate in tissues lacking normal spatial cues, or in tissues subjected to certain conditions. The methods and constructs described herein enable multiple cell types to be identified simultaneously in the same sample.

### BRIEF SUMMARY OF THE DISCLOSURE

Compositions and methods for regulating gene expression in a plant are provided. Compositions comprise a novel nucleotide sequence, and active fragments and variants thereof, for a promoter active in egg cells and/or embryonic cells of a plant. Embodiments of the disclosure also include DNA constructs comprising the promoter operably linked to a heterologous nucleotide sequence of interest, wherein the promoter is capable of driving expression of the nucleotide sequence in an egg cell-preferred and/or embryonic cell-preferred manner. Such compositions find use in, for example, methods for expressing a heterologous nucleotide sequence in a plant; detection of specific cell types in the ovule and targeted ablation of specific cell types and any combination thereof. Embodiments of the disclosure further provide expression vectors, plants, plant cells and seeds having stably incorporated into their genomes a DNA construct as described above.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 (FIG. 1A and FIG. 1B) demonstrates the microscopic evaluation of unpollinated maize kernels from PHP46361 ears showing egg cell-specific expression of ZsGreen when operably linked to the ZM-DD45 promoter. FIG. 1A and FIG. 1B—dissected maize kernel exposing the ovule and embryo sac. FIG. 1A is a two-color fluorescent image showing a ZsGreen fluorescent egg cell at the base of the embryo sac. Red color is intrinsic weak autofluorescence from the ovular tissues and the embryo sac. FIG. 1B is high magnification image of FIG. 1A showing detail of the ZsGreen positive egg cell.

FIG. 2 (FIG. 2A and FIG. 2B) demonstrates the expression pattern of ZsGreen operably linked to the ZM-DD45 promoter at the globular embryo stage of development in maize. At this stage it is highly reduced compared to that seen at the egg stage (FIG. 1A and FIG. 1B). No expression was observed at the later stages of development. FIG. 2A and FIG. 2B—dissected maize kernel exposing the ovule and embryo. FIG. 2A is a two-color fluorescent image showing a weakly fluorescent ZsGreen-positive embryo (arrow) at the base of the embryo sac. Blue color is intrinsic weak autofluorescence from the ovular tissues and embryo sac of the kernel. FIG. 2B is high magnification image of FIG. 2A showing detail of the young globular embryo which shows weak ZsGreen positive expression.

FIG. 3 (FIG. 3) demonstrates the expression pattern of ZsGreen operably linked to the ZM-DD45 promoter in a mature maize embryo, 8 days after pollination. No ZM-DD45-ZsGreen expression is observed at this stage or in the later stages of embryo development. FIG. 3 is a maize embryo dissected from the kernel. FIG. 3 is a two-color fluorescent image showing a lack of ZsGreen fluorescence in the embryo. Blue color is intrinsic weak autofluorescence, mostly from the cell walls, normally viewed when using a near-UV fluorescent DAPI filter set.

FIG. 4 (FIG. 4) illustrates the microscopic evaluation of kernels from PHP46360 ears indicating that the AT-DD45 promoter expressed very similarly to the maize DD45 promoter in maize kernels. DS-RED EXPRESS operably linked to the AT-DD45 was expressed in egg cells from unpollinated kernels. No expression was observed from AT-DD65 or AT-DD31 promoters. FIG. 4—dissected pre-fertilized maize kernel exposing the ovule, embryo sac (arrow) and egg. FIG. 4 is a two-color fluorescent image showing a fluorescent DsRed Express-positive egg at the base of the embryo sac. Blue color is intrinsic weak autofluorescence from the ovular tissues and embryo sac of the kernel.

FIG. 5 (FIG. 5) shows expression of DS-RED EXPRESS when operably linked to the AT-DD45 promoter (PHP46360) detected in an early embryo, 5 days post-pollination. No expression was observed from AT-DD65 or AT-DD31. FIG. 5 is a dissected maize kernel exposing the embryo sac and embryo. FIG. 5 is a two-color fluorescent image showing a fluorescent DsRed Express-positive embryo at the base of the embryo sac. Blue color is intrinsic weak autofluorescence from the ovular tissues and embryo sac of the kernel.

FIG. 6 (FIG. 6) shows motifs (highlighted) shared between the AT-DD45 and ZM-DD45 promoters.

FIG. 7 (FIG. 7A and FIG. 7B) demonstrates the expression pattern of event Php49807#2 AT-DD45:BARNASE—Triple label (DD2:ZsGreen) in EGS maintainer line php47029#21 in Arabidopsis ovules. Reference images exhibiting normal post-fertilization embryo-sacs wherein the egg cell, central cell and synergids can be visually identified and differentiated. FIG. 7A and FIG. 7B are three-color fluorescent images showing a fluorescent DsRed-positive egg/zygote and ZsGreen-positive synergids at the micropylar end of the embryo sac, and the AmCyan-positive central cell.

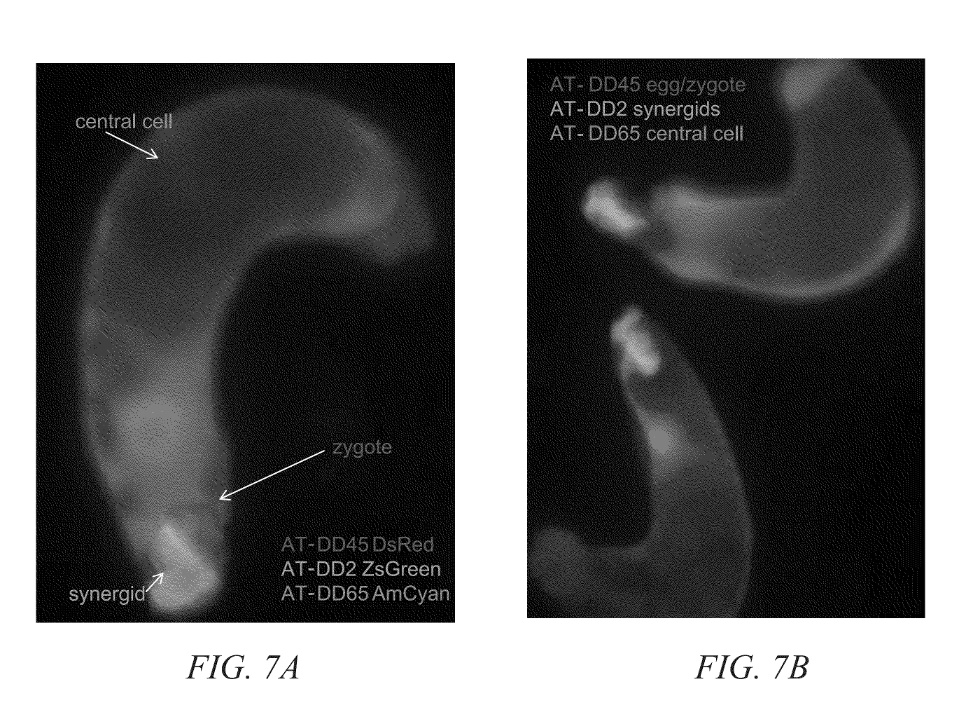

FIG. 8 (FIG. 8A and FIG. 8B) demonstrates the expression pattern of event Php49807#2 DD45:BARNASE—DD2:ZsGreen-DD45:DsRed-DD65:AmCyan in ovules of Arabidopsis EGS maintainer line php47029#21, wherein the egg cell was successfully ablated and persistent synergid and endosperm appear normal. FIG. 8A is a differential interference contrast (DIC) image of an Arabidopsis ovule overlayed with FIG. 8B. FIG. 8B is three-color fluorescent image showing a fluorescent ZsGreen-positive synergid and the AmCyan-positive central cell, the zygote (DsRed) is absent.

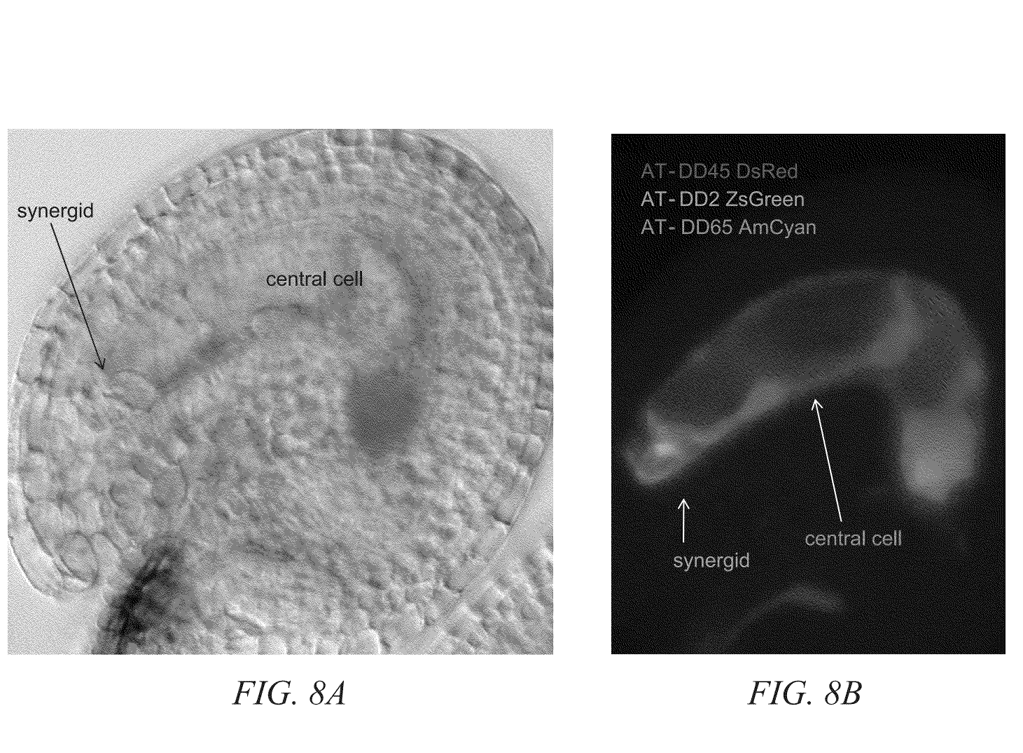

FIG. 9 (FIG. 9A, FIG. 9B and FIG. 9C) demonstrates the expression pattern of event Php49807#3 DD45:BARNASE—DD2:ZsGreen-DD45:DsRed-DD65:AmCyan in EGS maintainer line php47029#41, wherein the expression of barnase resulted in a highly enlarged and deformed zygote and synergid. FIG. 9A is a three-color fluorescent image of an Arabidopsis embryo sac showing a fluorescent DsRed-positive zygote, ZsGreen-positive synergid and the AmCyan-positive central cell. FIG. 9B and FIG. 9C are separate grayscale images of the synergid and zygote from FIG. 9A.

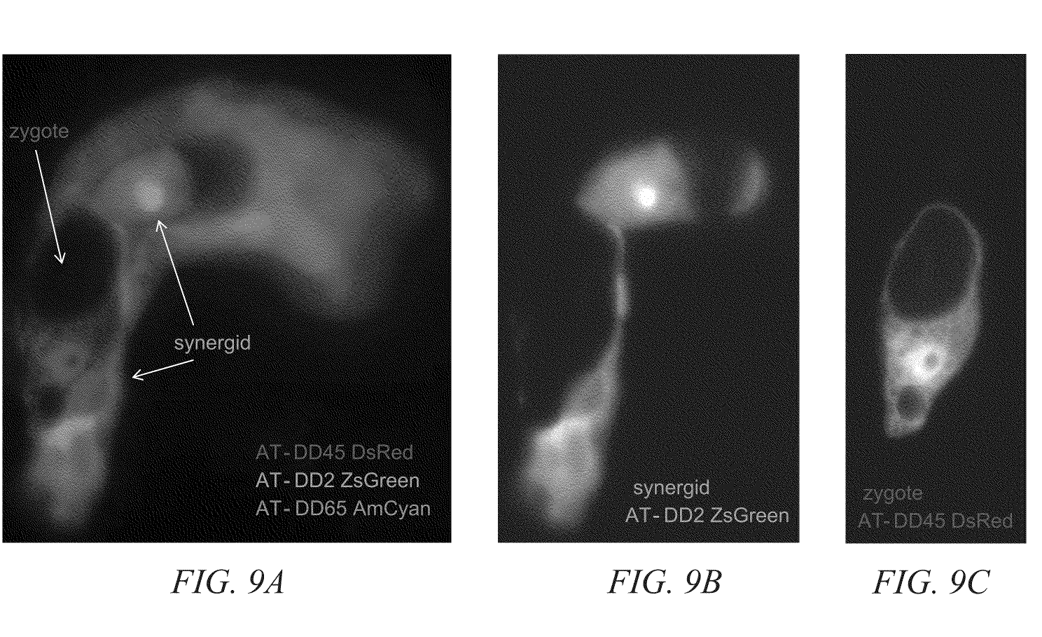

FIG. 10 (FIG. 10A, FIG. 10B, FIG. 10C and FIG. 10D) demonstrates the expression pattern of event Php50939 AT-RKD1:BARNASE—Triple label (AT-DD45:DsRed_AT-DD31:ZsYellow_AT-DD65:AmCyan) Arabidopsis ovules in EGS maintainer line php47029, exhibiting: fairly normal post-fertilization embryo-sacs with healthy zygotes, synergids and central cells/endosperm. FIG. 10A is a differential interference contrast (DIC) image of an Arabidopsis ovule overlayed with FIG. 10B. FIG. 10B, FIG. 10C, and FIG. 10D are three-color fluorescent images showing a ZsYellow-positive synergid, DsRed-positve zygote and the AmCyan-positive central cell.

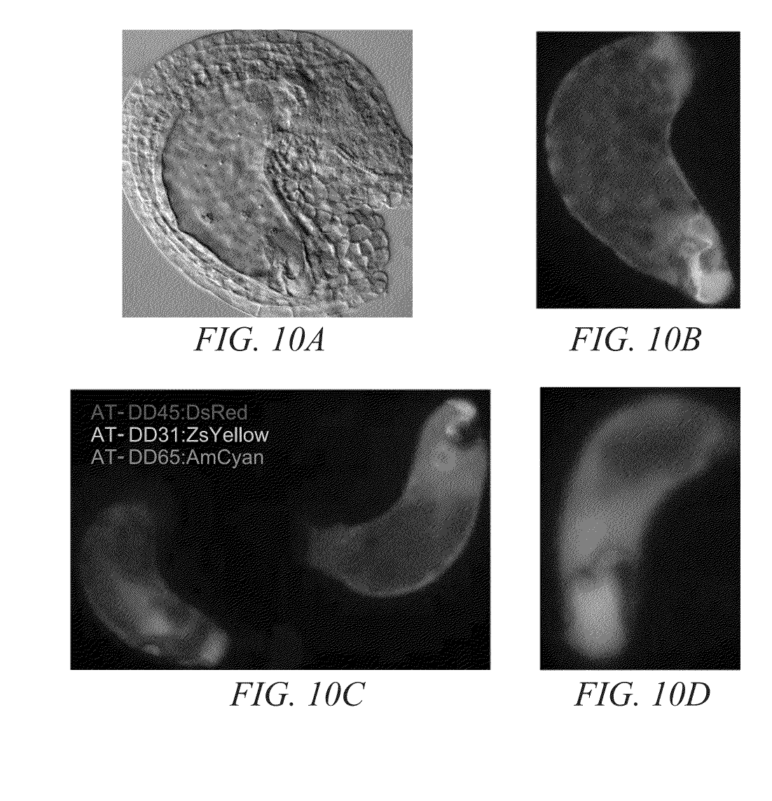

FIG. 11 (FIG. 11A, FIG. 11B and FIG. 11C)—Arabidopsis ovules that demonstrate the expression pattern of event Php50940 AT-RKD2:BARNASE—Triple label (AT-DD45:DsRed_AT-DD31:ZsYellow_AT-DD65:AmCyan) in EGS maintainer line php47029#51, exhibiting: a normal embryo-sac (FIG. 11A), orno synergids (FIG. 11B). FIG. 11C shows the endosperm developing in the absence of an embryo, indicating that it is possible to ablate the egg/zygote and still maintain endosperm development in the absence of the zygotic embryo. FIG. 11A, FIG. 11B, and FIG. 11C are three-color fluorescent images showing a ZsYellow-positive synergid, DsRed-positve zygotes and AmCyan-positive central cells.

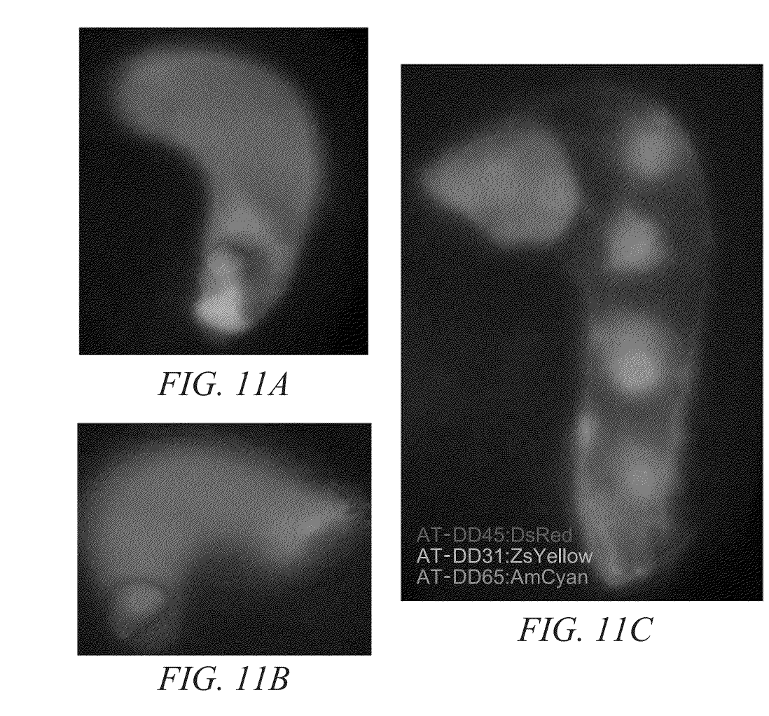

FIG. 12 (FIG. 12) demonstrates the expression pattern of event Php50940 AT-RKD2:BARNASE—Triple label (AT-DD45:DsRed_AT-DD31:ZsYellow_AT-DD65:AmCyan) in EGS maintainer line php47029#54, exhibiting the development of endosperm in the absence of a embryo (This shows that it is possible to ablate the egg/zygote and maintain endosperm development). Fluorescent image of 2 Arabidopsis embryo sacs. The embryo sac at left has numerous endosperm nuclei in its' central cell (AT-DD65:AmCyan) and at its' micropylar end (arrow) is a remnant of the embryo or zygote (AT-DD45:DsRed). Under normal conditions this embryo should be much more fully developed, at the heart-shaped stage. The smaller embryo sac at right has numerous endosperm nuclei (cyan), but is lacking an embryo altogether (arrow). Synergids would have been lost by this late stage and are expected to be present.

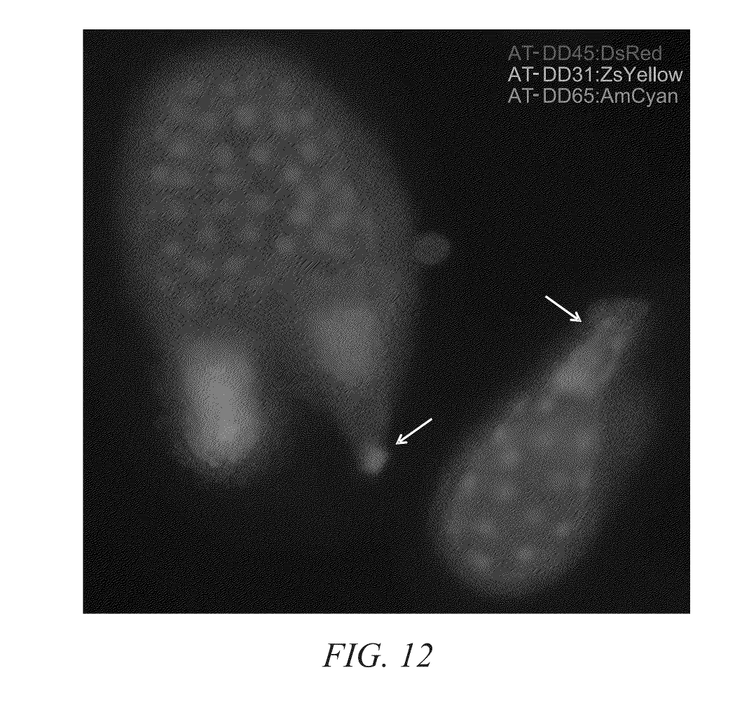

### DETAILED DESCRIPTION

The present disclosures now will be described more fully hereinafter with reference to the accompanying drawings, in which some, but not all embodiments of the disclosures are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

### Promoter Polynucleotides

Compositions and methods are provided drawn to plant promoters and methods of their use. In certain embodiments, the promoters drive expression in a manner that is cell type-preferred, cell type-specific, tissue-preferred or tissue-specific. The compositions provided herein comprise nucleotide sequences for an egg cell-preferred and/or embryonic cell-preferred promoter designated ZM-DD45 as set forth in SEQ ID NO: 34. In particular, isolated nucleic acid molecules are provided comprising the nucleotide sequence set forth in SEQ ID NO: 34, and active fragments and variants thereof. The compositions further comprise DNA constructs comprising a nucleotide sequence for the ZM-DD45 promoter or active fragment or variant thereof operably linked to a heterologous polynucleotide of interest.

In seed plants, the ovule is the structure that gives rise to and contains the female reproductive cells. It consists of three parts: The integument forming its outer layer, the nucellus (or megasporangium) and the funiculus. The nucellus produces the megasporocyte which will undergo meiosis to form the megaspore. Thus, as used herein, the ovule is composed of diploid tissue that gives rise to the haploid tissue of the female gametophyte. The female gametophyte or “egg sac” is comprised of four unique cell types: one egg cell, a central cell with two polar nuclei, two synergids and three or more antipodal cells. Upon fertilization, the egg cell (zygote) divides to form a proembryo in which apical and basal cells form wherein apical cells become the embryo. Cell division of the proembryo leads to the globular stage wherein tissue differentiation is evident and the epidermis begins to appear. Following the globular stage is the heart stage in which the two cotyledons become evident (dicots). While in monocots, a torpedo stage develops with a single cotyledon. The embryonic cells are now organized into an embryo proper with an apical meristem, radical, and cotyledon(s). The endosperm is formed from the fertilization of the second sperm and the two polar nuclei. The endosperm divides rapidly to fill the central cell and becomes the nutritive tissue for the developing embryo. In cotyledonous angiosperms, the mature embryo forms with a large cotyledon(s) and the endosperm becomes absorbed during embryogenesis. In endospermic angiosperms, such as maize, the endosperm is retained and becomes the main storage tissue for the seed. Early embryo development in maize is proembryo-transitional-coleoptilar. Later embryo development is simply labeled as 1-6 embryo stages according to W. Sheridan in Mutants of Maize. Differentiation of embryo proper into scutellum, embryonic axis and first leaf primordium occurs during transitional through stage 1 of embryo development.

As used herein, a “plant promoter” is a promoter capable of initiating transcription in plant cells whether or not its origin is a plant cell. In certain embodiments, plant promoters can preferentially initiate transcription in certain tissues, such as leaves, roots, seeds, or developmental growth stages, such as zygote, torpedo, early embryonic, globular embryo or late globular embryo. Such plant promoters are referred to as “tissue-preferred” or “cell type-preferred”. Promoters which initiate transcription only in certain tissue are referred to as “tissue-specific”. A “cell type-specific” promoter primarily drives expression in certain cell types in one or more organs, for example, vascular cells in roots or leaves or individual cell types within the ovule such as egg cells or embryonic cells.

The regulatory sequences provided herein, or variants or fragments thereof, when operably linked to a heterologous nucleotide sequence of interest can drive egg cell-preferred or embryonic cell-preferred expression of the heterologous nucleotide sequence in the reproductive tissue of the plant expressing this construct. The term “egg cell-preferred expression” or “initiates transcription in an egg cell-preferred manner” means that expression of the heterologous nucleotide sequence is most abundant in the egg cell of the ovule tissue. While some level of expression of the heterologous nucleotide sequence may occur in other plant tissue types, expression occurs most abundantly in the egg cell tissue. Likewise, “embryonic cell-preferred expression” or “initiates transcription in an embryonic cell-preferred manner” means that expression of the heterologous nucleotide sequence is most abundant in the embryonic cells in the ovule tissue. While some level of expression of the heterologous nucleotide sequence may occur in other plant tissue types, expression occurs most abundantly in the embryonic cell tissue. As used herein, the term “embryonic cells” refers to early embryonic cells, globular embryonic cells, late globular embryonic cells, or any other cells at the embryonic stage of development.

As used herein, the terms “promoter”, “promoter polynucleotide”, or “transcriptional initiation region” mean a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. It is recognized that having identified the nucleotide sequences for the promoter regions disclosed herein, it is within the state of the art to isolate and identify further regulatory elements in the 5′ untranslated region upstream from the particular promoter regions identified herein. Additionally, chimeric promoters may be provided. Such chimeras include portions of the promoter sequence fused to fragments and/or variants of heterologous transcriptional regulatory regions. Thus, the promoter regions disclosed herein can comprise upstream regulatory elements such as, those responsible for tissue and temporal expression of the coding sequence, enhancers and the like. In the same manner, the promoter elements, which enable expression in the desired tissue such as reproductive tissue, can be identified, isolated, and used with other core promoters to confer egg cell or embryonic cell-preferred expression. In this aspect of the disclosure, “core promoter” is intended to mean a promoter without promoter elements.

As used herein, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. In the context of the present disclosure a post-transcriptional regulatory element may include elements that are active following transcription initiation, for example translational and transcriptional enhancers, translational and transcriptional repressors and mRNA stability determinants.

The regulatory elements or variants or fragments thereof, of the promoters provided herein may be operatively associated with heterologous regulatory elements or promoters in order to modulate the activity of the heterologous regulatory element. Such modulation includes enhancing or repressing transcriptional activity of the heterologous regulatory element, modulating post-transcriptional events or either enhancing or repressing transcriptional activity of the heterologous regulatory element and modulating post-transcriptional events. For example, one or more regulatory elements of the present disclosure, or active fragments or variants thereof, may be operatively associated with constitutive, inducible, or tissue specific promoters or fragment thereof, to modulate the activity of such promoters within desired tissues in plant cells.

The promoter sequences provided herein can be modified to provide for a range of expression levels of the heterologous nucleotide sequence. Thus, less than the entire promoter region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of promoter deletions if, for example, there is a negative regulatory element (for a repressor) that is removed during the truncation process. Generally, at least about 20 nucleotides of an isolated promoter sequence will be used to drive expression of a nucleotide sequence.

It is recognized that to increase transcription levels, enhancers may be utilized in combination with the promoter regions of the disclosure. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Modifications of the isolated promoter sequences of the present disclosure can provide for a range of expression of the heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The promoter sequences provided herein include nucleotide constructs that allow initiation of transcription in a plant. In specific embodiments, the ZM-DD45 promoter sequences, or active fragments or variants thereof, allow initiation of transcription in a cell type-preferred manner. More particularly ZM-DD45, or active fragments or variants thereof, allows initiation of transcription in an egg cell-preferred or in an embryonic cell-preferred manner. Thus, the compositions provided herein include DNA constructs comprising a nucleotide sequence of interest operably linked to a ZM-DD45 promoter, or active fragments or variants thereof, which initiates expression in a plant, particularly in an egg cell-preferred or embryonic cell-preferred manner. A sequence comprising the ZM-DD45 promoter region is set forth in SEQ ID NO: 34.

Compositions include the nucleotide sequences for the native ZM-DD45 promoter, and active fragments and variants thereof. Such promoter sequences are useful for expressing any polynucleotide of interest. The ZM-DD45 promoter, or active fragments or variants thereof, expresses preferentially in the egg cells and embryonic cells. In specific embodiments, the promoter sequences are useful for expressing polynucleotides of interest in an embryonic cell-preferred or in an egg cell-preferred manner. The nucleotide sequences of the disclosure also find use in the construction of expression vectors for subsequent expression of a heterologous nucleotide sequence in a plant of interest or as probes for the isolation of other egg cell-preferred or embryonic cell-preferred promoters. In particular, expression constructs are provided comprising the ZM-DD45 promoter nucleotide sequence set forth in SEQ ID NO: 34, or active fragments or variants thereof, operably linked to a nucleotide sequence of interest. The ZM-DD45 promoter and active variants and fragments thereof which direct transcription in a cell-preferred manner as discussed in detail elsewhere herein, is particularly desirable for the expression of sequences of interest which promote apospory and adventitious embryony and other means for generating self-reproducing plants in crops, including but not limited to maize and similar species.

Substantially purified nucleic acid compositions comprising the promoter polynucleotides or active fragments or variants thereof are also provided. An “isolated” or “purified” nucleic acid molecule or biologically active portion thereof is substantially free of other cellular material or culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. The promoter sequences disclosed herein may be isolated from the 5′ untranslated region flanking their respective transcription initiation sites.

Fragments and variants of the disclosed promoter nucleotide sequences further provided. In particular, fragments and variants of the ZM-DD45 promoter sequences of SEQ ID NO: 34 may be used in the DNA constructs provided herein. As used herein, the term “fragment” refers to a portion of the nucleic acid sequence. Fragments of a ZM-DD45 promoter sequence may retain the biological activity of initiating transcription. More particularly fragments of ZM-DD45 may retain the biological activity of initiating transcription in an egg cell-preferred or embryonic cell-preferred manner. Alternatively, fragments of a nucleotide sequence that are useful as hybridization probes may not necessarily retain biological activity. Fragments of a nucleotide sequence for the ZM-DD45 promoter region may range from at least about 6 nucleotides, about 8 nucleotides, about 10 nucleotides, about 12 nucleotides, about 15 nucleotides, about 20 nucleotides, about 30 nucleotides, about 40 nucleotides, about 50 nucleotides, about 100 nucleotides and up to the full length of SEQ ID NO: 34. A biologically active portion of a ZM-DD45 promoter can be prepared by isolating a portion of the ZM-DD45 promoter sequence of the disclosure, and assessing the promoter activity of the portion.

As used herein, the term “variants” is intended to mean sequences having substantial similarity with a promoter sequence disclosed herein. A variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, such as, for example, with polymerase chain reaction (PCR) and hybridization techniques as outlined herein.

Variant nucleotide sequences also include synthetically derived nucleotide sequences, such as those generated, for example, by using site-directed mutagenesis. Generally, variants of a particular nucleotide sequence of the embodiments will have at least 40%, 50%, 60%, 65%, 70%, 75%, 80%, 85%, 90%, 91%, 92%, 93%, 94%, to 95%, 96%, 97%, 98%, 99% or more sequence identity to that particular nucleotide sequence as determined by sequence alignment programs described elsewhere herein using default parameters. Biologically active variants are also encompassed by the embodiments. Biologically active variants include, for example, the native promoter sequences of the embodiments having one or more nucleotide substitutions, deletions or insertions. Promoter activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook,” herein incorporated by reference in its entirety. Alternatively, levels of a reporter gene such as green fluorescent protein (GFP) or yellow fluorescent protein (YFP) or the like produced under the control of a promoter fragment or variant can be measured. See, for example, Matz, et al., (1999) Nature Biotechnology 17:969-973; U.S. Pat. No. 6,072,050, herein incorporated by reference in its entirety; Nagai, et al., (2002) Nature Biotechnology 20(1):87-90.

Variant nucleotide sequences also encompass sequences derived from a mutagenic and recombinogenic procedure such as DNA shuffling. With such a procedure, one or more different ZM-DD45 promoter nucleotide sequences can be manipulated to create a new ZM-DD45 promoter. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety.

The nucleotide sequences provided herein can be used to isolate corresponding sequences from other organisms, including other plants or other monocots. In this manner, methods such as PCR, hybridization and the like can be used to identify such sequences based on their sequence homology to the sequences set forth herein. Sequences isolated based on their sequence identity to the entire ZM-DD45 sequences set forth herein or to fragments thereof are encompassed by the present disclosure. Thus, isolated sequences that have egg cell-preferred or embryonic cell-preferred promoter activity and which hybridize under stringent conditions to the ZM-DD45 promoter sequences, disclosed herein or to fragments thereof, are encompassed by the present disclosure.

In general, sequences that have promoter activity and hybridize to the promoter sequences disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65; and Pearson, et al., (1994) Meth. Mol. Biol. 24:307-331, herein incorporated by reference in their entirety. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403, herein incorporated by reference in its entirety, are based on the algorithm of Karlin and Altschul, (1990) supra. BLAST nucleotide searches can be performed with the BLASTN program, score=100, word length=12, to obtain nucleotide sequences homologous to a nucleotide sequence encoding a protein of the disclosure. BLAST protein searches can be performed with the BLASTX program, score=50, word length=3, to obtain amino acid sequences homologous to a protein or polypeptide of the disclosure. To obtain gapped alignments for comparison purposes, Gapped BLAST (in BLAST 2.0) can be utilized as described in Altschul, et al., (1997) Nucleic Acids Res. 25:3389, herein incorporated by reference in its entirety. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, the web site for the National Center for Biotechnology Information on the World Wide Web at ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2 and the BLOSUM62 scoring matrix; or any equivalent program thereof. As used herein, “equivalent program” is any sequence comparison program that, for any two sequences in question, generates an alignment having identical nucleotide or amino acid residue matches and an identical percent sequence identity when compared to the corresponding alignment generated by GAP Version 10.

The GAP program uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see, Henikoff and Henikoff, (1989) Proc. Natl. Acad. Sci. USA 89:10915, herein incorporated by reference in its entirety).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tₘ for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

### Expression Cassettes

The nucleotide sequences disclosed herein, as well as variants and fragments thereof, are useful in the genetic manipulation of any plant. The ZM-DD45 promoter sequences or active fragments or variants thereof are useful in this aspect when operably linked with a heterologous nucleotide sequence whose expression is to be controlled to achieve a desired phenotypic response. The term “operably linked” means that the transcription of the heterologous nucleotide sequence is under the influence of the promoter sequence. In this manner, the nucleotide sequences for the promoters disclosed herein may be provided in expression cassettes along with heterologous nucleotide sequences of interest for expression in the plant of interest, more particularly for expression in the reproductive tissue of the plant.

In one embodiment of the disclosure, expression cassettes will comprise a transcriptional initiation region comprising the promoter nucleotide sequence disclosed herein, or active variants or fragments thereof, operably linked to a heterologous nucleotide sequence. Such an expression cassette can be provided with a plurality of restriction sites for insertion of the nucleotide sequence to be under the transcriptional regulation of the regulatory regions. The expression cassette may additionally contain selectable marker genes as well as 3′ termination regions.

The expression cassette can include, in the 5′-3′ direction of transcription, a transcriptional initiation region (i.e., a promoter, or active variant or fragment thereof, as disclosed herein), a translational initiation region, a heterologous nucleotide sequence of interest, a translational termination region and optionally, a transcriptional termination region functional in the host organism. The regulatory regions (i.e., promoters, transcriptional regulatory regions and translational termination regions) and/or the polynucleotide of the embodiments may be native/analogous to the host cell or to each other. Alternatively, the regulatory regions and/or the polynucleotide of the embodiments may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus or the promoter is not the native promoter for the operably linked polynucleotide.

The termination region may be native with the transcriptional initiation region, may be native with the operably linked DNA sequence of interest, may be native with the plant host, or may be derived from another source (i.e., foreign or heterologous to the promoter, the DNA sequence being expressed, the plant host or any combination thereof). Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639, herein incorporated by reference in their entirety.

The expression cassette comprising the sequences of the present disclosure may also contain at least one additional nucleotide sequence for a gene to be cotransformed into the organism. Alternatively, the additional sequence(s) can be provided on another expression cassette. In some embodiments, the expression cassette may contain additional promoters operably linked to additional heterologous polynucleotides of interest. For example, expression cassettes disclosed herein may have 1, 2, 3, 4, 5, 6, 7, 8, 9 or 10 additional promoters operably linked to heterologous polynucleotides of interest.

Where appropriate, the nucleotide sequences whose expression is to be under the control of the egg cell-preferred or embryonic cell-preferred promoter sequences disclosed herein and any additional nucleotide sequence(s) may be optimized for increased expression in the transformed plant. That is, these nucleotide sequences can be synthesized using plant preferred codons for improved expression. See, for example, Campbell and Gowri, (1990) Plant Physiol. 92:1-11, herein incorporated by reference in its entirety, for a discussion of host-preferred codon usage. Methods are available in the art for synthesizing plant-preferred genes. See, for example, U.S. Pat. Nos. 5,380,831, 5,436,391 and Murray, et al., (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize Adhl intron (Kyozuka, et al., (1991) Mol. Gen. Genet. 228:40-48; Kyozuka, et al., (1990) Maydica 35:353-357) and the like, herein incorporated by reference in their entirety.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in the expression cassettes of the present disclosure. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other polynucleotides of interest that could be employed include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

As used herein, “vector” refers to a DNA molecule such as a plasmid, cosmid or bacterial phage for introducing a nucleotide construct, for example, an expression cassette, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

### Heterologous Polynucleotides of Interest

A “heterologous nucleotide sequence” is a sequence that is not naturally occurring with the promoter sequence of the disclosure. While this nucleotide sequence is heterologous to the promoter sequence, it may be homologous (native) or heterologous (foreign) to the plant host.

Heterologous coding sequences expressed by a ZM-DD45 promoter, or active fragments or variants thereof, disclosed herein may be used for varying the phenotype of a plant or plant progeny by preferentially expressing a polynucleotide of interest in egg cells or embryonic cells. Various changes in phenotype are of interest including modifying expression of a gene in a plant, preferentially expressing marker polynucleotides in tissues of interest, targeted cell ablation, female sterility, initiating adventitious embryony or apomixis and the like. These results can be achieved by the expression of a heterologous nucleotide sequence of interest encoding an appropriate gene product under the transcriptional control of the promoter polynucleotides disclosed herein.

In specific embodiments, the heterologous nucleotide sequence of interest is a plant or plant-derived sequence whose expression level is increased in the plant or plant part. Tissue-preferred expression as provided by the ZM-DD45 promoter, or active fragments or variants thereof, can target the alteration in expression to plant parts and/or growth stages of particular interest, such as developing ovule cell types, particularly egg cells or embryonic cells within the ovule. These changes can result in a change in phenotype of the transformed plant. In certain embodiments, the expression patterns of egg cell-preferred promoters or embryonic cell-preferred promoters, such as the ZM-DD45 promoter, or active fragments or variants thereof, are particularly useful for screens for female sterility, apomixis, adventitious embryony, artificial apospory, detection of specific cell types, targeted cell ablation and the generation of self reproducing hybrids.

General categories of nucleotide sequences of interest for the present disclosure include, for example, those genes involved in information, such as zinc fingers, those involved in communication, such as kinases and those involved in housekeeping, such as heat shock proteins. Other categories of transgenes include genes for inducing expression of exogenous products such as enzymes, cofactors and hormones from plants and other eukaryotes as well as prokaryotic organisms. Still other categories of transgenes include reporter genes that allow visualization or detection of individual cell types within the ovule including, but not limited to, egg cells and embryonic cells. Categories of transgenes may also include genes for ablating cells, such as cytotoxins. It is recognized that any gene of interest can be operably linked to the promoter of the disclosure and expressed in the plant.

When the ZM-DD45 promoter disclosed herein, or an active fragment or variant thereof, is operably linked to a heterologous polynucleotide of interest encoding a reporter gene, detection of the expressed protein may be detected in a seed, plant or plant cell. Thus, reporter genes disclosed herein may allow visualization or detection of individual cell types including egg cells and embryonic cells. Expression of the linked protein can be detected without the necessity of destroying tissue. By way of example without limitation, the promoter can be linked with detectable markers including a β-glucuronidase or uidA gene (GUS), which encodes an enzyme for which various chromogenic substrates are known (Jefferson, et al., (1986) Proc. Natl. Acad. Sci. USA 83:8447-8451); maize-optimized phosphinothricin acetyl transferase (moPAT); chloramphenicol acetyl transferase; alkaline phosphatase; a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988); Ludwig, et al., (1990) Science 247:449); a p-lactamase gene (Sutcliffe, (1978) Proc. Nat'l. Acad. Sci. U.S.A. 75:3737), which encodes an enzyme for which various chromogenic substrates are known (e.g., PADAC, a chromogenic cephalosporin); a xylE gene (Zukowsky, et al., (1983) Proc. Nat'l. Acad. Sci. U.S.A. 80:1101), which encodes a catechol dioxygenase that can convert chromogenic catechols; an α-amylase gene (Ikuta, et al., (1990) Biotech. 8:241); a tyrosinase gene (Katz, et al., (1983) J. Gen. Microbiol. 129:2703), which encodes an enzyme capable of oxidizing tyrosine to DOPA and dopaquinone, which in turn condenses to form the easily detectable compound melanin a green fluorescent protein (GFP) gene (Sheen, et al., (1995) Plant J. 8(5):777-784); a lux gene, which encodes a luciferase, the presence of which may be detected using, for example, X-ray film, scintillation counting, fluorescent spectrophotometry, low-light video cameras, photon counting cameras or multiwell luminometry (Teeri, et al., (1989) EMBO J. 8:343); DS-RED or DS-RED EXPRESS (Matz, et al., (1999) Nature Biotech. 17:969-973, Bevis, et al., (2002) Nature Biotech 20:83-87, Haas, et al., (1996) Curr. Biol. 6:315-324); Zoanthus sp. yellow fluorescent protein (ZsYellow) that has been engineered for brighter fluorescence (Matz, et al., (1999) Nature Biotech. 17:969-973, available from BD Biosciences Clontech, Palo Alto, Calif., USA, catalog no. K6100-1); ZsGreen; AmCyan; and cyan florescent protein (CYP) (Bolte, et al., (2004) J. Cell Science 117:943-954 and Kato, et al., (2002) Plant Physiol 129:913-942).

Reporter genes may be selected taking into account color of the encoded detectable protein. For example, in case a green fluorescent protein is chosen, it may be GFP, EGFG, AcGFP, TurboGFP, Emerald, Azani Green or ZsGreen. In case a blue fluorescent protein is chosen, it may be EBFP, tagBFP, Sapphire or T-Sapphire. In case a cyan fluorescent protein is chosen, it may be ECFP, mCFP, Cerulean, CyPet, AmCyan, AmCyanl, Midori-Ishi Cyan or mTFP1 (Teal). In case a yellow fluorescent protein is chosen, it may be EYFP, Topaz, Venus, mCitrine, Ypet, PhiYFP, tagYFP, ZsYellow, ZsYello1 or mBanana. In case a red or orange fluorescent protein is chosen, it may be Kusabira Orange, mOrange, dTomato, dTomato-Tandem, DsRed, DsRed2, DsRed-Expresss (T1), DsRed Express, DsRed Express2, tagRFP, DSRed-Monomer, mTangerine, mStrawberry, AsRed2, mRFP1, Jred, mCherry, HcRed1, mRaspberry, HcRed-Tandem, mPlum or AQ143. In some embodiments, expression cassettes and plants disclosed herein comprise multiple promoters expressing different colors of detectable fluorescent proteins. For example, different colors of fluorescent proteins could be used to simultaneously detect and differentiate cell types within the ovule. If different colors of fluorescent proteins are expressed within the ovule, fluorescent protein color may be selected such that cell types can be easily differentiated from each other. For example, a red fluorophore could be selected for expression in the egg cell, a blue fluorophore in the central cell, and a green fluorophore in the synergid cells.

The expression cassettes described herein may further contain other tissue-preferred promoters operably linked to a heterologous polynucleotide of interest. Alternatively, the expression cassettes described herein may be transformed into a plant comprising separate expression cassettes comprising tissue-preferred promoters operably linked to a heterologous polynucleotide of interest. In certain embodiments, expression cassettes are provided comprising promoters that preferentially express a different color fluorophore in at least 2, at least 3 or all four of the cell types in the ovule (e.g. egg cell, central cell, synergid cells, and antipodal cells). In specific embodiments, each fluorophore is selected in order to provide adequate differentiation between cell types for detection and differentiation of individual cell types within the ovule. Promoter polynucleotides used for preferential expression in egg cells include, but are not limited to: ZM-DD45 (SEQ ID NO: 34), AT-DD45 (SEQ ID NO: 10), AT-RKD1 PRO, AT-RKD2 PRO, AT-RKD3 PRO and AT-RKD4 PRO. Promoter polynucleotides used for preferential expression in central cells include, but are not limited to: ZM-FEM2 (SEQ ID NO: 30) and AT-DD65 (SEQ ID NO: 43). Promoter polynucleotides used for preferential expression in antipodal cells include, but are not limited to: AT-DD1 (SEQ ID NO: 41). Promoter polynucleotides used for preferential expression in synergid cells include, but are not limited to: AT-DD31 (SEQ ID NO: 42), AT-DD2 (SEQ ID NO: 20), Egg Apparatus Specific Enhancer (EASE) (SEQ ID NO: 19). Other examples of cell type-preferred promoters can be found, for example, in Steffen, (2007) Plant J. 51(2):281-292.

The constructs and methods disclosed herein can be used for, inter alia, characterization and assessment of cell-specific ablation constructs; tracking of cell fates under typical growth conditions, or tracking of cell fate changes upon system perturbations (ablation, adventitious embryony, etc). The compositions and methods may be used to identify proto-embryos developing from callus tissue. The methods and constructs could also be used for cell sorting, for transcript profiling with additional promoter isolation, or for proteomic or metabolomic profiling. There may be additional applications for targeted manipulations of egg cells or developing embryos.

In other embodiments, the heterologous polynucleotides of interest disclosed herein may encode proteins capable of causing cell ablation. As used herein, the term “cell ablation” refers to targeted damage of a specific cell. In some embodiments, cell ablation results in the death of the cell or damage to the cell such that the cell no longer divides or differentiates. Preferential ablation of the egg cell without adversely affecting the central cell or synergids could be a tool for the production of female sterile plants. Proteins capable of causing cell ablation include cytotoxins such as barnase (Yoshida, (2001) Methods Enzymol 341:28-41), Dam Methylase (see, Barras, (1989) Trends in Genetics 5:139-143), ADP ribosylase (see, Fan, (2000) Curr. Opin. Struct. Biol., 10:680-686), nucleases, or any other protein or nucleic acid capable of cell ablation.

As set forth above, in certain embodiments, egg cell ablation could be used to produce female sterile plants. Female sterile male inbred lines could be interplanted with male sterile female lines to create hybrid seed without the necessity of human intervention, such as detasseling or removing male inbred rows after pollination.

The ability to stimulate organogenesis and/or somatic embryogenesis may be used to generate an apomictic plant. Apomixis can cause any genotype, regardless of how heterozygous, to breed true. It is a reproductive process that bypasses female meiosis and syngamy to produce embryos genetically identical to the maternal parent. With apomictic reproduction, progeny of specially adapted or hybrid genotypes could maintain their genetic fidelity throughout repeated life cycles. In addition to fixing hybrid vigor, apomixis can make possible commercial hybrid production in crops where efficient male sterility or fertility restoration systems for producing hybrids are not available. Apomixis can make hybrid development more efficient. The apomixis process also simplifies hybrid production and increases genetic diversity in plant species with good male sterility. Furthermore, apomixis may be advantageous under stress (drought, cold, high-salinity, etc.) conditions where pollination may be compromised.

In certain embodiments, the expression cassettes disclosed herein can be combined with expression cassettes comprising nucleic acid molecules encoding transcription factors, for example RKD transcriptions factors (i.e., RKD2), capable of inducing an egg cell-like state from somatic cells of the ovule. Such RKD transcription factors include those set forth in any one of SEQ ID NO: 18, 20, 22, 24 and 32 and biologically active variants and fragments thereof. Further provided are the polynucleotides (SEQ ID NO: 17, 19, 21, 23 and 31) encoding these various RKD transcription factors and active variant and fragments thereof.

For example, expression cassettes can comprise the promoter polynucleotides, or active fragments or variants thereof, disclosed herein operably linked to a heterologous polynucleotide encoding a cytotoxin, wherein expression of the cytotoxin ablates the egg cell or embryonic cell such that development of the embryo from an egg cell does not take place. In such a case, a second expression cassette could be provided wherein a polynucleotide encoding a transcription factor (i.e., RKD transcription factor), capable of inducing an egg cell-like state from somatic cells of the ovule, is operably linked to an ovule tissue-preferred promoter active in a somatic ovule cell of a plant. The combination of egg cell or embryonic cell ablation with expression of a transcription factor in a somatic ovule cell could induce an egg cell-like state in a somatic cell while preserving normal development of the central cell and endosperm. See, U.S. Provisional Patent Application Ser. No. ______, entitled Methods and Compositions for Modulating Expression or Activity of an RKD Polypeptide a Plant, filed concurrently herewith and herein incorporated by reference in its entirety.

Expression of a marker polynucleotide (i.e., a fluorescent marker polynucleotide) from an egg cell-preferred or embryonic cell-preferred promoter disclosed herein, or active fragments or variants thereof, could allow detection and/or visualization of an egg cell-like state induced in a somatic cell. For example, expression of a cytotoxin from an egg cell-preferred or embryonic cell-preferred promoter disclosed herein, or fragments or variants thereof, along with expression of a transcription factor such as an RKD2 transcription factor in somatic ovule tissues can cause ablation of the egg cell or embryonic cell along with inducing an egg cell-like state in a somatic tissue, as described above. Further, expression of a fluorescent marker polynucleotide in the same plant operably linked to an egg cell-preferred or embryonic cell-preferred promoter disclosed herein, or fragments or variants thereof, can allow detection and/or visualization of the egg cell-like state induced in the somatic cells. The fluorescent marker polynucleotides and cytotoxins described above operably linked to an egg cell-preferred or embryonic cell-preferred promoter disclosed herein, or fragments or variants thereof, and the polynucleotides encoding a transcription factor capable of inducing an egg cell-like state in somatic cells of the ovule operably linked to an ovule tissue-preferred promoter can be located on three separate nucleic acid molecules or combined on two nucleic acid molecules or combined on a single nucleic acid molecule.

Expression cassettes, plants and seeds are further provided that comprise polynucleotides of interest encoding both cytotoxins and fluorescent markers operably linked to promoters, such as the ZM-DD45 promoter or active fragments or variants thereof, for cell type-preferred expression in the egg cells or embryonic cells of a plant. By expressing cytotoxins mediating cell ablation along with fluorescent markers, the fate of individual cell types and effectiveness of cell ablation can be monitored. For example, when a cytotoxin is specifically expressed under the control of an egg cell-specific promoter, expression of a fluorescent marker also under the control of an egg cell-specific promoter can report the efficacy of the cytotoxin by detecting the viability of the egg cell. Further, in the same scenario, by operably linking polynucleotides encoding fluorescent proteins to other cell type-specific promoters such as central cell-specific promoters, the effect of an egg cell-expressed cytotoxin on the central cell can also be detected.

For example, expression cassettes comprising a polynucleotide encoding barnase under the control of the ZM-DD45 promoter, or active fragments or variants thereof, along with a polynucleotide encoding DS-Red under the control of the ZM-DD45 promoter, or active fragments or variants thereof, allows for visual confirmation and detection of ablated egg cells in the ovule. In certain embodiments, expression cassettes comprising multiple detectable marker polynucleotides (i.e., encoding different colors of fluorophores) can be provided that allow simultaneous detection of different cell types within the ovule. In particular embodiments, expression cassettes comprising multiple detectable marker polynucleotides as set forth above include but are not limited to: ZM-DD45:BARNASE-Triple label (ZM-DD45:DsRed AT-DD2:ZsGreen AT-DD65:AmCyan).

Proteins encoded by the heterologous polynucleotides of interest disclosed herein may be assembled by intein-mediated trans-splicing. See, for example, Gils, (2008) Plant Biotech. Journal 6:226-235 and Kempe, (2009) Plant Biotech. Journal 7:283-297, herein incorporated by reference in their entirety. For example, expressed barnase fragments may be assembled by intein-mediated trans-splicing. The intein-fused barnase fragments, or polynucleotides encoding the fragments, may be located in different parental plants and may be under control of different developmentally regulated or cell type-preferred promoters. Said fragments may be brought together upon hybridization to form a cytotoxic product as the result of intein-mediated trans-splicing. The use of different promoters with different yet partially overlapping expression patterns may confine barnase activity to the required tissue in a more precise way than by using the same tissue-specific promoters to drive the expression of both barnase fragments.

In another embodiment, the ZM-DD45 promoter, or an active fragment or variant thereof, is used to express transgenes that modulate organ development, stem cell development, initiation and development of the apical meristem, such as the Wuschel (WUS) gene; see, U.S. Pat. Nos. 7,348,468 and 7,256,322 and US Patent Application Publication Number 2007/0271628 published Nov. 22, 2007; Laux, et al., (1996) Development 122:87-96 and Mayer, et al., (1998) Cell 95:805-815. Modulation of WUS is expected to modulate plant and/or plant tissue phenotype including cell growth stimulation, organogenesis, and somatic embryogenesis. WUS may also be used to improve transformation via somatic embryogenesis. Expression of Arabidopsis WUS can induce stem cells in vegetative tissues, which can differentiate into somatic embryos (Zuo, et al., (2002) Plant J 30:349-359). Also of interest in this regard would be a MYB118 gene (see, U.S. Pat. No. 7,148,402), MYB115 gene (see, Wang, et al., (2008) Cell Research 224-235), BABYBOOM gene (BBM; see, Boutilier, et al., (2002) Plant Cell 14:1737-1749) or CLAVATA gene (see, for example, U.S. Pat. No. 7,179,963); LEC1; RKD transcription factors; orthologs thereof or combinations of these CDSs with this promoter or other PTU.

The heterologous nucleotide sequence operably linked to the ZM-DD45 promoter and its related biologically active fragments or variants disclosed herein may be an antisense sequence for a targeted gene. The terminology “antisense DNA nucleotide sequence” is intended to mean a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having 70%, 80%, 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides or greater may be used. Thus, the promoter sequences disclosed herein may be operably linked to antisense DNA sequences to reduce or inhibit expression of a native protein in the plant.

“RNAi” refers to a series of related techniques to reduce the expression of genes (see, for example, U.S. Pat. No. 6,506,559, herein incorporated by reference in its entirety). Older techniques referred to by other names are now thought to rely on the same mechanism, but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced. The ZM-DD45 promoters of the embodiments may be used to drive expression of constructs that will result in RNA interference including microRNAs and siRNAs.

The expression cassettes and vectors comprising the ZM-DD45 promoter of the present disclosure operably linked to a heterologous nucleotide sequence of interest can be used to transform any plant. In this manner, genetically modified plants, plant cells, plant tissue, seed, root and the like can be obtained.

### Plants

The ZM-DD45 promoter sequence disclosed herein, as well as active variants and fragments thereof, are useful for genetic engineering of plants, e.g. for the production of a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a transgene of interest, the regeneration of a population of plants resulting from the insertion of the transgene into the genome of the plant and selection of a particular plant characterized by insertion into a particular genome location. An event is characterized phenotypically by the expression of the transgene. At the genetic level, an event is part of the genetic makeup of a plant. The term “event” also refers to progeny produced by a sexual cross between the transformant and another plant wherein the progeny include the heterologous DNA.

As used herein, the term plant includes whole plants, plant organs (e.g., leaves, stems, roots, etc.), plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the disclosure, provided that these parts comprise the introduced polynucleotides.

The present disclosure may be used for transformation of any plant species, including, but not limited to, monocots and dicots. Examples of plant species include corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present disclosure include, for example, pines such as loblolly pine (Pinus taeda), slash pine (Pinus elliotii), ponderosa pine (Pinus ponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis). In specific embodiments, plants of the present disclosure are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The methods and compositions of the disclosure involve introducing a polypeptide or polynucleotide into a plant and plants having stably incorporated into their genome the polynucleotides and expression cassettes disclosed herein. As used herein, “introducing” is intended to mean presenting to the plant the polynucleotide or polypeptide in such a manner that the sequence gains access to the interior of a cell of the plant. The methods of the disclosure do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

A “stable transformation” is a transformation in which the nucleotide construct introduced into a plant integrates into the genome of the plant and is capable of being inherited by the progeny thereof. “Transient transformation” means that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 2000/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference in their entirety.

The cells that have been transformed may be grown into plants in accordance with conventional ways. See, for example, McCormick, et al., (1986) Plant Cell Reports 5:81-84. These plants may then be grown, and either pollinated with the same transformed strain or different strains and the resulting progeny having constitutive or cell type-preferred expression of the desired phenotypic characteristic identified, based on the promoter polynucleotide selected. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, or active fragments or variants thereof, for example, an expression cassette disclosed herein, stably incorporated into their genome.

### Methods of Use

Methods for using the promoter polynucleotides disclosed herein are provided. Such methods comprise stably incorporating in the genome of a plant or plant cell a heterologous polynucleotide of interest operably linked to a promoter polynucleotide as described herein (i.e. SEQ ID NO: 34) or active variants or fragments thereof.

Depending on the polynucleotide of interest operably linked to the promoter polynucleotides as described herein, the transgenic plants, plant cells or seeds may have a change in phenotype, including, but not limited to, tissue-specific fluorescent marker expression, targeted cell ablation, female sterility, initiation of adventitious embryony or apomixis, and the like.

i. Detection and Differentiation of Cell Types

In specific embodiments, the promoter polynucleotides provided herein are used to preferentially express at least one heterologous polynucleotide of interest in a cell, wherein detection of the heterologous polynucleotide of interest identifies the type of cell. The heterologous polynucleotide of interest can be preferentially expressed in a plant cell, wherein detection of the heterologous polynucleotide of interest identifies the type of plant cell. The heterologous polynucleotide of interest operably linked to the promoter polynucleotides described herein can be any marker polynucleotide, including a fluorescent marker polynucleotide encoding a fluorophore, wherein detection of the marker identifies the cell type. In specific embodiments, methods are provided to detect the presence of an egg cell or embryonic cell, wherein ZM-DD45 is operably linked to a marker polynucleotide encoding a fluorophore. Detection of such a fluorophore would thereby identify the presence of an egg cell or embryonic cell. Detection of fluorescent markers or fluorophore can be effected by detecting fluorescence emission after excitation at a proper wavelength, chemiluminescence or light absorbance. Such detection can be achieved by detecting fluorescence emission using a fluorescence microscope. In certain embodiments, the detection of fluorescent markers is quantitative. Immunocytochemistry using antibodies targeting the heterologous polynucleotide may be used in conjuction with bright field, fluorescence or electron microscopy to detect promoter expression. In situ hybridization may also be used to identify heterologous or native nucleotide expression.

Detection of said heterologous polynucleotide of interest in a cell can identify the type of cell based on the promoter polynucleotide of the disclosure operably linked to the heterologous polynucleotide of interest. For example, in certain embodiments, expression cassettes are provided comprising ZM-DD45, or active fragments or variants thereof, operably linked to a fluorescent marker polynucleotide and another ovule cell type-specific promoter also linked to a fluorescent marker polynucleotide, wherein detection of each encoded fluorophore identifies the presence of both an egg cell and corresponding to other cell types within the ovule.

Thus, methods are provided herein for the simultaneous detection of different cell types within an ovule. In some embodiments, the detection and differentiation of different cell types within the ovule of a plant can be achieved using fluorescent marker polynucleotides operably linked to tissue-preferred promoter polynucleotides disclosed herein. For example, in certain embodiments, expression cassettes stably incorporated into the genome of a plant comprise the ZM-DD45 promoter operably linked to a first fluorescent marker polynucleotide and further comprise the ZM-FEM2 promoter operably linked to a second fluorescent marker polynucleotide whose expressed fluorophore can readily be distinguished from the fluorophore encoded by the first fluorescent marker polynucleotide. In specific embodiments, the ZM-DD45 promoter is operably linked to a red fluorescent marker polynucleotide and ZM-FEM2 is operably linked to a cyan fluorescent marker polynucleotide. In such an embodiment, expression of the red fluorescent marker preferentially in the egg cell, and expression of the cyan fluorescent marker preferentially in the central cell allows simultaneous detection of each cell type and differentiation of the egg cell from the central cell. In some embodiments the absence of detection of a marker (i.e., fluorophore) expressed by the heterologous polynucleotide of interest operably linked to a promoter polynucleotide of the disclosure indicates a specific cell type is not present.

Methods disclosed herein for detection and differentiation of cell types within the ovule of a plant can be achieved prior to fertilization, after fertilization or at any other stage of development. Expression of a marker polynucleotide (i.e., a fluorescent marker polynucleotide) from an egg cell-preferred or embryonic cell-preferred promoter disclosed herein, or active fragments or variants thereof, could allow detection and/or visualization of an egg cell-like state induced in a somatic cell. For example, expression of a cytotoxin from an egg cell-preferred or embryonic cell-preferred promoter disclosed herein, or fragments or variants thereof, along with expression of a transcription factor, such as an RKD2 transcription factor, in somatic ovule tissues can cause ablation of the egg cell or embryonic cell along with inducing an egg cell-like state in a somatic tissue, as described elsewhere herein. Further, expression of a fluorescent marker polynucleotide in the same plant operably linked to an egg cell-preferred or embryonic cell-preferred promoter disclosed herein, or fragments or variants thereof, can allow detection and/or visualization of the egg cell-like state induced in the somatic cells.

ii. Cell-Preferred Ablation

Cell-preferred or cell-specific ablation is useful in initiating adventitious embryony, female sterility, apomixis, synthetic apospory, female sterility and other methods for producing self-reproducing hybrids. For example, by specifically ablating the egg cell, fertilization of the central cell can still occur along with some degree of endosperm development. Thus, prevention of the formation of the zygotic embryo by egg cell ablation allows for the possibility of adventitious embryo formation from non-reduced cells in the ovule. For example, expression of a heterologous polynucleotide encoding a cytotoxin operably linked to a promoter polynucleotide, or active fragments or variants thereof, disclosed herein can cause egg cell or embryonic cell ablation such that development of the embryo from an egg cell does not take place. In such a case, a second polynucleotide operably linked to an ovule tissue-preferred promoter active in a somatic ovule cell outside of the embryo sac of a plant can further be expressed, encoding a transcription factor (i.e., RKD2), capable of inducing an egg cell-like state from somatic cells of the ovule. The combination of egg cell or embryonic cell ablation with expression of a transcription factor in a somatic ovule cell could induce an egg cell-like state in a somatic cell while preserving normal development of the central cell and endosperm.

In specific embodiments, the promoter polynucleotides disclosed herein are used to preferentially ablate specific cell types within a plant or plant cell. For example, the promoter polynucleotides disclosed herein can be operably linked to a heterologous polynucleotide of interest encoding a cytotoxin, wherein the cytotoxin preferentially ablates a specific cell type. As used herein “preferential ablation” or “preferentially ablates” refers to ablation that primarily occurs in the target cell with minimum influence on non-target cell types. For example, “egg cell-preferred ablation” refers to ablation primarily occurring in the egg cell, and “embryonic cell-preferred ablation” refers to ablation primarily occurring in the embryonic cells. Ablation of the egg cells and embryonic cells can be detected by the expression of a polynucleotide of interest encoding a marker polynucleotide (i.e., fluorescent marker polynucleotide) operably linked to the ZM-DD45 promoter, or an active fragment or variant thereof. Further, the effect of egg cell-preferred or embryonic cell-preferred ablation on other cell types within the ovule can be detected by the expression of a marker polynucleotide (i.e., fluorescent marker polynucleotide) from a promoter that preferentially or specifically expresses the marker polynucleotide in a target cell type within the ovule such as the central cell, synergid cells, or antipodal cells, as described in detail elsewhere herein. Thus, egg cell-preferred ablation or embryonic cell-preferred ablation would ablate the egg cells or embryonic cells, respectively, with a minimal effect on other cell types within the ovule.

In some embodiments, the ZM-DD45 promoter, or active fragments or variants thereof, is operably linked to a heterologous polynucleotide of interest encoding a cytotoxin, for example barnase, that is preferentially expressed in the egg cell of the ovule, thereby ablating the egg cell. Preferential ablation of the egg cell by expression of a cytotoxin from the ZM-DD45 promoter, or active fragments or variants thereof, can cause female sterility of the resulting plant. Thus, female sterile plants are provided produced by the methods disclosed herein.

Further provided are expression cassettes and plants for the expression of fragments of a cytotoxin, such as barnase. Cytotoxin fragments may be brought together upon fertilization or hybridization to form a cytotoxic product as the result of intein-mediated trans-splicing. For example, different barnase fragments may be expressed in different plants under the control of different developmentally regulated or cell type-preferred promoters, such as the ZM-DD45 promoter, or active fragments or variants thereof. When the plants are crossed, the barnase fragments may be brought together to form a functional cytotoxic barnase protein. Other promoters include but are not limited to: Female: AT-DD45 promoter; AT-RKD1 promoter; AT-RKD2 promoter; AT-RKD3 promoter; AT-RKD4 promoter. Male: LAT52 promoter (pollen); inducible promoters constitutive promoters pollen preferred promoters such as PG47, P95 and P67 promoters. Anther promoters such as Ms45Pro, Ms26Pro, Bs7Pro, 5126 Pro.

Methods of the disclosure include providing expression cassettes comprising one or more than one cell type-specific or cell type-preferred promoter operably linked to a cytotoxin as described elsewhere herein and/or operably linked to polynucleotides of interest encoding detectable markers as described herein. Simultaneous cell type-specific expression or cell type-preferred expression of both cytotoxins and detectable markers can allow for ablation of specific cell types and subsequent detection of ablated cell types. For example, expression of barnase under the control of the ZM-DD45 promoter, or active fragments or variants thereof, simultaneously with expression of DS-Red under the control of the ZM-DD45 promoter, or active fragments or variants thereof, allows for visual confirmation and detection of the ablated cell type. In such a case, the barnase could specifically ablate the egg cell, while the absence of DS-Red expression may indicate successful ablation of egg cells or embryonic cells in the ovule. As set forth above, expression cassettes comprising multiple detectable marker polynucleotides (i.e., encoding different colors of fluorophores) can be provided that allow simultaneous detection of different cell types within the ovule. Further, cytotoxins can be provided under the control of the promoter polynucleotides described herein simultaneously with multiple detectable marker polynucleotides that allow for detection of ablated cell types and concurrent detection of other cell types within the ovule. Such a method can be used to determine the effects of cell type-preferred or cell type-specific expression of cytotoxins on non-target cells within the ovule.

In some embodiments, expression cassettes are introduced into a plant comprising an expression cassette, also referred to as maintenance vectors, capable of expressing barstar. Expression of barstar cancels the effects of barnase and is able to prevent cell ablation in specific cell types, even in the presence of barnase. Maintenance vectors capable of expressing barstar could exist in the genetic background of a plant or they could be introduced along with the expression cassettes described herein comprising the promoter polynucleotides of the disclosure. Thus, plants are provided produced by the methods disclosed herein comprising a maintenance vector capable of expressing barstar and further comprising an expression cassette as described elsewhere herein.

TABLE 1

| | | | POLYNUCLEOTIDE/ |
|---|---|---|---|
| | | | POLYPEPTIDE |
| SEQ ID. | NAME | DESCRIPTION | (PN/PP) |
| SEQ ID NO: 1 | AT-NUC1 PRO | OVULE TISSUE- | PN |
| | (AT4G21620) | PREFERRED | |
| | | PROMOTER | |
| SEQ ID NO: 2 | ALT- AT-NUC1 | OVULE TISSUE- | PN |
| | PRO | PREFERRED | |
| | (AT4G21620) | PROMOTER | |
| SEQ ID NO: 3 | AT-CYP86C1 | OVULE TISSUE- | PN |
| | (AT1G24540) | PREFERRED | |
| | | PROMOTER | |
| SEQ ID NO: 4 | ALT- AT- | OVULE TISSUE- | PN |
| | CYP86C1 | PREFERRED | |
| | | PROMOTER | |
| SEQ ID NO: 5 | AT-PPM1 PRO | OVULE TISSUE- | PN |
| | AT5G49180 | PREFERRED | |
| | | PROMOTER | |
| SEQ ID NO: 6 | AT-EXT PRO | OVULE TISSUE- | PN |
| | AT3G48580 | PREFERRED | |
| | | PROMOTER | |
| SEQ ID NO: 7 | AT-GILT1 PRO | OVULE TISSUE- | PN |
| | AT4G12890 | PREFERRED | |
| | | PROMOTER | |
| SEQ ID NO: 8 | AT-TT2 PRO | OVULE TISSUE- | PN |
| | AT5G35550 | PREFERRED | |
| | | PROMOTER | |
| SEQ ID NO: 9 | AT-SVL3 PRO | OVULE TISSUE- | PN |
| | | PREFERRED | |
| | | PROMOTER | |
| SEQ ID NO: 10 | AT-DD45 PRO | EGG CELL-PREFERRED | PN |
| | | PROMOTER | |
| SEQ ID NO: 11 | ATRKD1 | CDNA OF RKD | PN |
| | FULL LENGTH | POLYPEPTIDE | |
| | CDNA | | |
| SEQ ID NO: 12 | ATRKD1 | RKD POLYPEPTIDE | PP |
| | AMINO ACID | | |
| | NM_101737.1 | | |
| SEQ ID NO: 13 | ATRKD2 | CDNA OF RKD | PN |
| | (AT1G74480) | POLYPEPTIDE | |
| | FULL LENGTH | | |
| | CDNA | | |
| | NM_106108 | | |
| SEQ ID NO: 14 | ATRKD2 | RKD POLYPEPTIDE | PP |
| | (AT1G74480) | | |
| | AMINO ACID | | |
| SEQ ID NO: 15 | ATRKD3 | CDNA OF RKD | PN |
| | (AT5G66990) | POLYPEPTIDE | |
| | FULL LENGTH | | |
| | CDNA | | |
| | NM_126099 | | |
| SEQ ID NO: 16 | ATRKD3 | RKD POLYPEPTIDE | PP |
| | (AT5G66990) | | |
| | AMINO ACID | | |
| | NP_201500.1 | | |
| SEQ ID NO: 17 | ATRKD4 | CDNA OF RKD | PN |
| | (AT5G53040) | POLYPEPTIDE | |
| | FULL LENGTH | | |
| | CDNA | | |
| SEQ ID NO: 18 | ATRKD4 | RKD POLYPEPTIDE | PP |
| | (AT5G53040) | | |
| | AMINO ACID | | |
| | NP_200116.1 | | |
| SEQ ID NO: 19 | EASE PRO | EGG CELL-PREFERRED | PN |
| | | PROMOTER | |
| SEQ ID NO: 20 | AT-DD2 PRO | EGG CELL-PREFERRED | PN |
| | | PROMOTER | |
| SEQ ID NO: 21 | AT-RKD1 PRO | EGG CELL-PREFERRED | PN |
| SEQ ID NO: 22 | AT-RKD2 PRO | EGG CELL-PREFERRED | PN |
| SEQ ID NO: 23 | BA-BARNASE- | DNA ENCODING | PN |
| | INT | CYTOTOXIC | |
| | | POLYPEPTIDE | |
| SEQ ID NO: 24 | DAM | DNA ENCODING | PN |
| | METHYLASE | CYTOTOXIC | |
| | | POLYPEPTIDE | |
| SEQ ID NO: 25 | DMETH N-TERM | OLIGONUCLEOTIDE | PN |
| SEQ ID NO: 26 | INTE-N | OLIGONUCLEOTIDE | PN |
| SEQ ID NO: 27 | INTE-C | OLIGONUCLEOTIDE | PN |
| SEQ ID NO: 28 | DMETH C-TERM | OLIGONUCLEOTIDE | PN |
| SEQ ID NO: 29 | ADP | DNA ENCODING | PN |
| | RIBOSYLASE | CTYOTOXIC | |
| | | POLYPEPTIDE | |
| SEQ ID NO: 30 | FEM2 | EMBRYO SAC- | PN |
| | | PREFERRED | |
| | | PROMOTER | |
| SEQ ID NO: 31 | ATRKD5 | CDNA OF RKD | PN |
| | AT4G35590; DNA; | POLYPEPTIDE | |
| | ARABIDOPSIS | | |
| | THALIANA | | |
| SEQ ID NO: 32 | AT-RKD5; | RKD POLYPEPTIDE | PP |
| | PRT; ARABIDOPSIS | | |
| | THALIANA | | |
| SEQ ID NO: 33 | AT1G24540 | OVULE TISSUE- | PN |
| | AT-CP450-1 PRO | PREFERRED | |
| | | PROMOTER | |
| SEQ ID NO: 34 | ZMDD45PRO; | PROMOTER | PN |
| | DNA; ZEA MAYS | | |
| SEQ ID NO: 35 | PCO659480 | OLIGONUCLEOTIDE | PN |
| | 5PRIMELONG; | | |
| | DNA; ZEA MAYS | | |
| SEQ ID NO: 36 | PCO659480 | OLIGONUCLEOTIDE | PN |
| | 3PRIMELONG; | | |
| | DNA; ZEA MAYS | | |
| SEQ ID NO: 37 | ZSGREEN5PRIME; | OLIGONUCLEOTIDE | PN |
| | DNA; ZOANTHUS SP | | |
| SEQ ID NO: 38 | ZSGREEN3PRIME; | OLIGONUCLEOTIDE | PN |
| | DNA; ZOANTHUS SP | | |
| SEQ ID NO: 39 | CYAN1 5PRIME; | OLIGONUCLEOTIDE | PN |
| | DNA; ANEMONIA | | |
| | MAJANO | | |
| SEQ ID NO: 40 | CYAN1 3PRIME; | OLIGONUCLEOTIDE | PN |
| | DNA; ANEMONIA | | |
| | MAJANO | | |
| SEQ ID NO: 41 | AT-DD1 PRO; | PROMOTER | PN |
| | DNA; | | |
| | ARABIDOPSIS | | |
| | THALIANA | | |
| SEQ ID NO: 42 | AT-DD31 PRO; | PROMOTER | PN |
| | DNA; | | |
| | ARABIDOPSIS | | |
| | THALIANA | | |
| SEQ ID NO: 43 | AT-DD65 PRO; | PROMOTER | PN |
| | DNA; | | |
| | ARABIDOPSIS | | |
| | THALIANA | | |
| SEQ ID NO: 44 | SORGHUM | PROMOTER - OVULE | PN |
| | BICOLOR OVULE | | |
| | SPECIFIC | | |
| | PROMOTER 1 | | |
| | (SB10G008120.1) | | |
| SEQ ID NO: 45 | PROMOTER | PROMOTER - OVULE | PN |
| | RICE OVULE | | |
| | CANDIDATE 1 | | |
| | (OS02G-51090) | | |
| SEQ ID NO: 46 | AT-RKD2 PRO | PROMOTER WITH | PN |
| | (AT1G74480) | PROPOSED TETOP | |
| | | SITES. OPTION 1 | |
| SEQ ID NO: 47 | AT-RKD2 PRO | PROMOTER WITH | PN |
| | (AT1G74480) | PROPOSED TETOP | |
| | | SITES. OPTION 2 | |
| SEQ ID NO: 48 | AT-RKD2 PRO | PROMOTER WITH | PN |
| | (AT1G74480) | PROPOSED TETOP | |
| | | SITES. OPTION 3 | |
| SEQ ID NO: 49 | BA-BASTAR; | CYTOTOXIC COGNATE | PN |
| | DNA; BACILLUS | REPRESSOR | |
| | AMYLOLIQUEFACIENS | | |
| SEQ ID NO: 50 | AT-RKD3 PRO; | PROMOTER | PN |
| | DNA; | | |
| | ARABIDOPSIS | | |
| | THALIANA | | |
| SEQ ID NO: 51 | AT-RKD4 PRO; | PROMOTER | PN |
| | DNA; | | |
| | ARABIDOPSIS | | |
| | THALIANA | | |
| SEQ ID NO: 52 | AT-RKD5 PRO; | PROMOTER | PN |
| | DNA; | | |
| | ARABIDOPSIS | | |
| | THALIANA | | |
| SEQ ID NO: 53 | AT-LAT52LP1 | PROMOTER | PN |
| | PRO; DNA; | | |
| | ARABIDOPSIS | | |
| | THALIANA | | |
| SEQ ID NO: 54 | AT-LAT52LP2 | PROMOTER | PN |
| | PRO; DNA; | | |
| | ARABIDOPSIS | | |
| | THALIANA | | |
| SEQ ID NO: 55 | AT-PPG1 PRO; | PROMOTER | PN |
| | DNA; | | |
| | ARABIDOPSIS | | |
| | THALIANA | | |
| SEQ ID NO: 56 | AT-PPG2 PRO; | PROMOTER | PN |
| | DNA; | | |
| | ARABIDOPSIS | | |
| | THALIANA | | |

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

### EXPERIMENTAL

### Example 1

### Identification of the ZM-DD45 Promoter

The Zm-DD45 gene was cloned from B73 genomic DNA by using PCR to amplify approximately 1.3 Kb upstream of the putative translational start using the PCR primer shown in SEQ ID NO: 35 and down through the putative promoter translational stop codon using primer shown as SEQ ID NO: 36. The PCR fragment was extracted from an agarose gel slice using Qiagen's QIAquick Gel Extraction Kit and cloned into Invitrogen's pCR2.1 TOPO Vector using manufacturer's instructions. This clone was used to subclone the ZM-DD45 promoter (SEQ ID NO: 34) into a transformation vector to drive the expression of the fluorescent reporter gene, ZS-GREEN1. This clone was designated PHP46361 and contained: ZM-DD45 PRO:ZS-GREEN1—UBIZM PRO:UBIZM 5′UTR:UBIZM INTRON:MO-PAT

A second construct containing the Arabidopsis DD45 promoter was designated PHP46360 and contained: AT-DD45 PRO:DS-RED EXPRESS—AT-DD31PRO:AC-GFP1—AT-DD65 PRO:AM-CYAN1. Approximately, ten single copy T0 maize plants for each construct were obtained through transformation of GS3/Gaspe flint lines. A GS3 male parent was used to cross onto the T0 plants to create T1 seed. Ten seeds from two T1 events from each construct were planted and seedlings were genotyped for the presence of the ZS-GREEN1 gene (SEQ ID NOS: 37 and 38) or for the presence of the CYAN1 gene (SEQ ID NOS: 39-40) using PCR. Transgenic null siblings were used as males to make crosses onto the transformed plants. Either unpollinated ears or 5DAP ears were harvested for microscopic examination.

### Example 2

### Microscopic Observation of Egg Cell-Specific Expression

Ears were kept on ice and individual kernels (unpollinated and 5DAP) were dissected from the ears and placed in PBS (pH7.2) on ice. Some kernels were fixed for long term storage, placed in 4% para-formaldehyde overnight at 4° C. then washes 3 times in PBS and stored at 4° C. Each kernel was then carefully sectioned, vertical or horizontal longitudinally, using an ophthalmic scalpel in order to obtain 100-300 μM thick slices with the intact embryo sac inside. These tissue slices were placed on glass slides in PBS and ready for microscopic observations.

Observations and images were taken with a Leica (Wetzlar, Germany) DMRXA epi-fluorescence microscope with a mercury light source. The Alexa 488 #MF-105 (exc. 486-500, dichroic 505LP, em. 510-530) fluorescent filter set was used to monitor ZsGreen fluorescence. Autofluorescence from the kernel tissues was also monitored using Cy3 #C-106250 (exc. 541-551, dichroic 560LP, em. 565-605) and DAPI #31013 (exc. 360-370, dichroic 380LP, em. 435-485) filter sets. All fluorescence filters sets were from Chroma Technology (Bellows Falls, Vt.). Images were captured with a Photometrics (Tucson, Ariz.) CoolSNAP HQ CCD. Camera and microscope were controlled, and images manipulated by Molecular Devices (Downingtown, Pa.) MetaMorph imaging software. Some final image manipulations were accomplished with Adobe Systems (San Jose, Calif.) Photoshop CS.

### Example 3

### ZM-DD45 Promoter Expresses Preferentially in Egg Cells

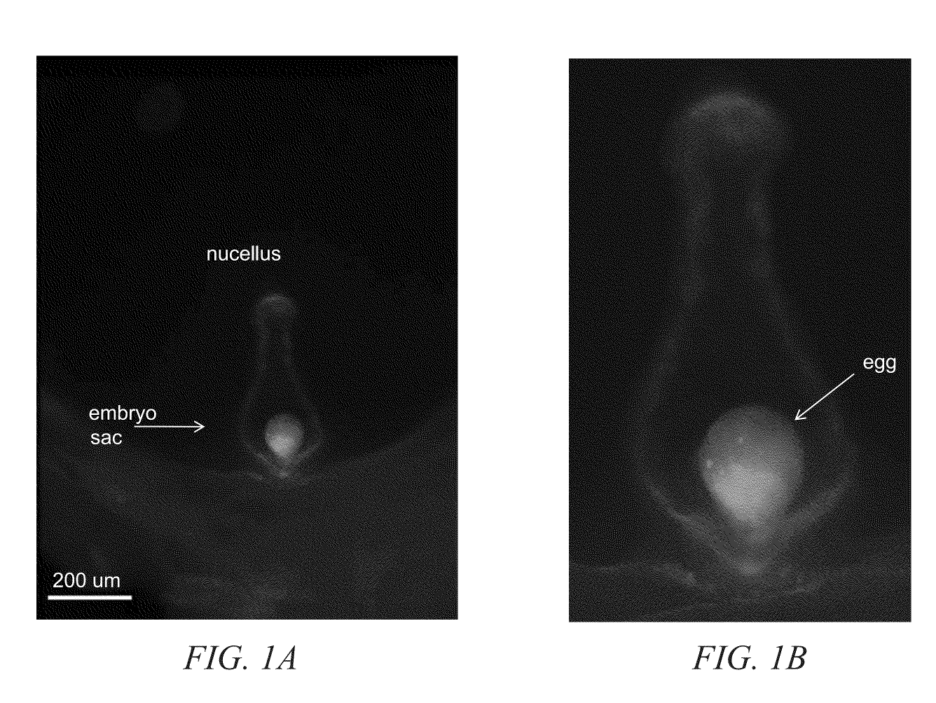

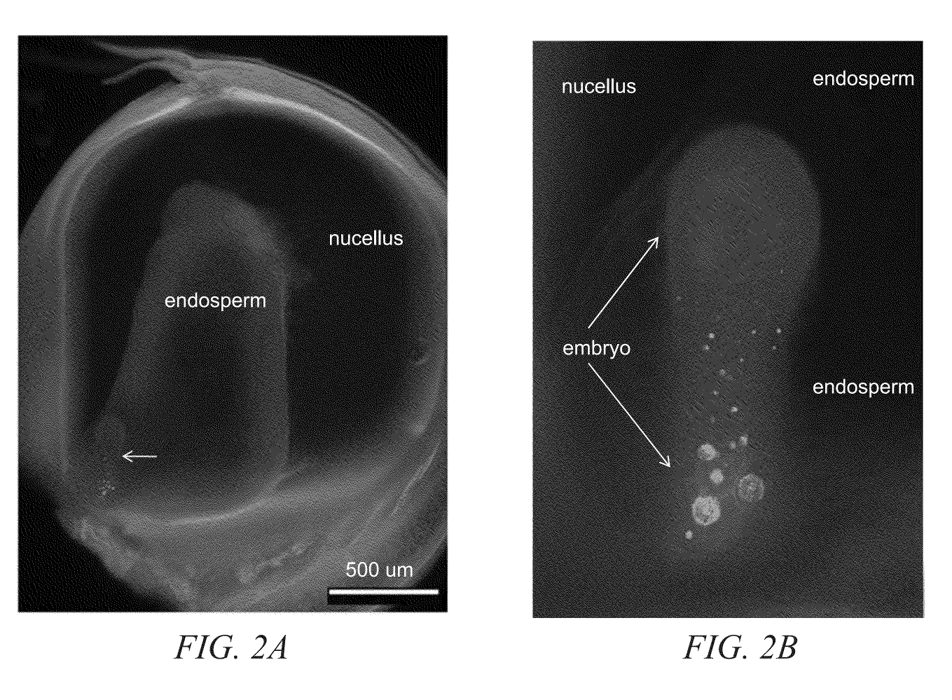

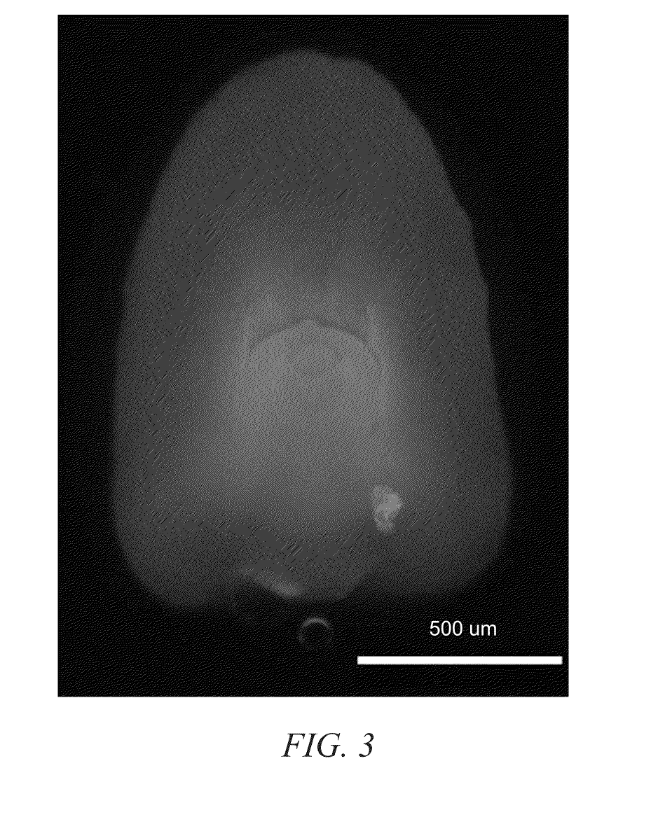

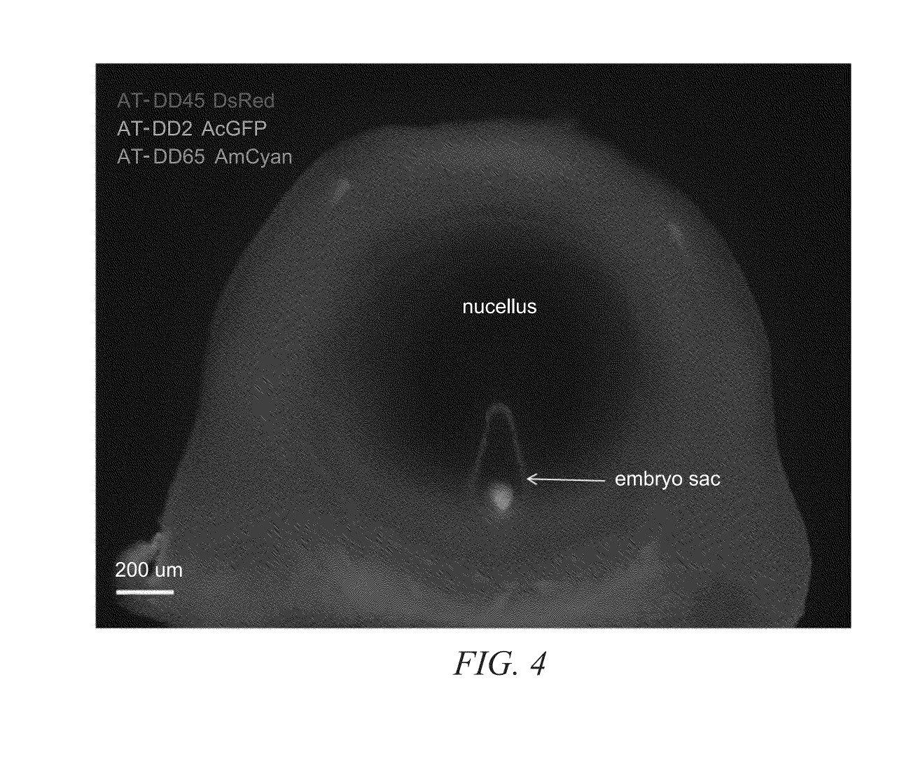

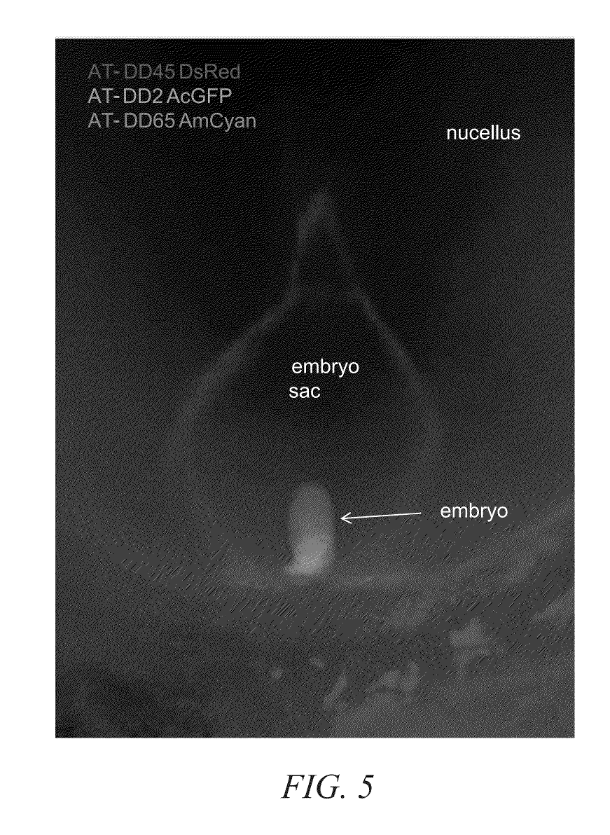

Microscopic evaluations of unpollinated kernels from PHP46361 ears revealed ZsGreen fluorescence in the egg cells only (FIG. 1). ZsGreen fluorescence was also detected in young embryos after pollination. By the globular embryo stage of development, the ZsGreen fluorescence is reduced or diluted (FIG. 2) and at later stages of embryo development the fluorescence cannot be detected (FIG. 3). These observations suggest that the ZM-DD45 promoter expresses specifically in egg cells and in early embryo development. Microscopic evaluations of kernels from PHP46360 ears showed that the AT-DD45 promoter expressed very similarly as the maize DD45 promoter in maize kernels. DS-RED EXPRESS fluorescence was detected only in egg cells from unpollinated kernels (FIG. 4). This fluorescence is also seen in early embryo development (FIG. 5) but begins to wane at the globular and later stages of embryo development.

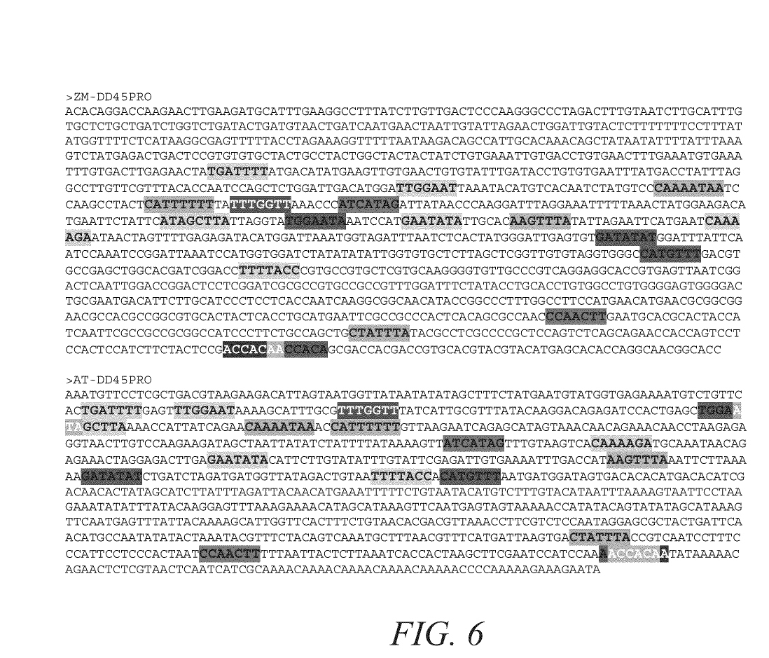

Both the Arabidopsis and the Maize DD45 promoters express specifically in the egg cell and in early embryo development and the Arabidopsis DD45 promoter maintains that expression pattern when expressed in maize. No significant similarity is found using BLAST between the sequence of the two promoters. However, using the PromoterReaper program (US Patent Application Publication Number 2010/0138952) eighteen motifs were found in common between the two promoter sequences, and some of these motifs are most likely involved in directing expression to the egg cell and early embryo (FIG. 6).

### Example 4

### Distinct Fluorescent Labeling of Cell Types within the Arabidopsis Egg Sac

This example describes the combination of multiple cell-type-specific promoters with distinct fluorescent proteins to individually label up to four different cell types in the egg sac. Up to four different Arabidopsis promoters are used:

(1) antipodal cell promoter AT-DD1 PRO; downregulated in dif1 (determinant infertile1) 1; At1g36340); SEQ ID NO: 41;

(2) synergid cell promoter AT-DD31 PRO; downregulated in dif1 (determinant infertile1) 31; At1g47470; SEQ ID NO: 42; or synergid cell promoter AT-DD2 PRO, SEQ ID NO: 10; Matz, et al., (1999) Nat Biotech 17(10):969-973; Erratum, (1999) Nat Biotech 17(12):1227-1227; Clontechniques (2003) XVIII(3):6-7; Clontechniques (2005) XX(1):5-7.

(3) egg cell promoter AT-DD45 PRO; downregulated in dif1 (determinant infertile1) 45; At2g21740; SEQ ID NO: 10; and
(4) central cell promoter AT-DD65 PRO; downregulated in dif1 (determinant infertile1) 65; At3g10890; SEQ ID NO: 43.

### See, Steffen, et al., (2007) Plant J. 51:281-292.

Each cell-type-specific promoter is operably linked to a polynucleotide encoding one of four distinct fluorescent proteins, with potentially similar colors spatially separated, to enhance unique detection: synergid promoter (DD31 PRO. DD2 PRO, or EASE PRO):green fluorescent protein; DD45 PRO:red fluorescent protein; DD65 PRO:cyan fluorescent protein; DD1 PRO:yellow fluorescent protein. Many possible new combinations can be produced.

These constructs or any partial combination (i.e., any two or more promoters driving expression of unique fluorescent proteins) would be useful for at least two purposes. The first is to report on cell-type-specific ablation/death in a transgenic or mutant plant. The second is to report adventitious creation of these cell types in other contexts. Such an outcome may arise in the successful or partially successful creation of adventitious embryony (a component of aposporous apomixis).

### Example 5

### Ablation of Specific Cell Types

Cell-type-specific promoters may be useful in constructs and methods designed to ablate certain cell types. Cell ablation to manipulate fertilization and/or seed development could include, for example, use of one or more of the cell type-specific promoters. Individual promoters would be particularly useful for cell ablation to prevent pollen tube attraction for fertilization (synergid ablation, DD31 or DD2); prevent sexual embryo formation (egg cell ablation, DD45, ZM-DD45, AT-RKD1, AT-RKD2), antipodal ablation (AT-DD1 or other antipodal promoters), and/or prevent endosperm formation (central cell ablation, ZM-FEM2, DD65). Additionally, the synergid, egg, or antipodal cell promoters could be useful for parthenogenesis. The egg and central cell promoters could be useful for zygote or early endosperm manipulations involving composition changes (oil, protein, carbohydrates) or disease/insect resistance. The egg cell promoter could be useful to induce recombinase enzymes (such as CRE or FLP) to remove or otherwise manipulate transgenes in maternal or paternal genomes. Meganucleases could be similarly controlled by promoters preferentially expressed in cell types within the ovule.

For example, it may be desirable to prevent formation of the zygotic embryo in developing seed. This would be useful, for example, in propagating hybrids and other favorable genotypes not easily reproduced by sexual means.

Arabidopsis promoter RKD2 (SEQ ID NO: 22) is used to specifically ablate egg cells in plant ovules. Analysis of this promoter, first identified by Koszegi, et al., (Koszegi, et al., Plant J 67:280-291), shows that it is specific to the egg cell and zygote/early embryo, and is not expressed in any other cell types. Using the RKD2 promoter to express a toxin (e.g., BARNASE; see, Beals and Goldberg, (1997) Plant Cell 9:1527-1545) would lead to egg cell ablation and prevent formation of the zygotic embryo. Since only the egg cell would be ablated, fertilization of the central cell should be possible along with some degree of endosperm development.

Prevention of the zygotic embryo is a component of a synthetic approach to self-reproducing plants. That is, the zygotic embryo is not formed, but an adventitious embryo is formed from non-reduced cells in the ovule. Prophetically, the adventitious embryo would develop so long as the central cell was fertilized and the endosperm co-developed in the ovule/seed.

Use of the RKD2 promoter is advantageous over the artificial EASE promoter disclosed in Yang, et al., ((2005) Plant Physiol 139(3):1421-1432). The EASE promoter in our analysis does not appear to be specific to the egg cell. Preliminary observations suggest that this promoter is either specific to the synergids or co-expressed in synergids and the egg cell. Ablation using a promoter with this expression pattern would prevent fertilization of the central cell because synergids are required for pollen tube attraction. Prophetically, an adventitious embryo would abort without co-development of the endosperm. In contrast, the specificity of the RKD2 promoter provides optimal control of expression of the toxin, driving egg cell ablation without disruption of other cell types in the embryo sac. This provides at least one advantage in that the nutritive endosperm is required for normal seed/embryo development.

### Example 6

### Generation of Transgenic Plants

Transgenic plant lines can be established via any transformation method, for example, Agrobacterium-mediated infection or particle bombardment.

i. Agrobacterium Mediated Transformation

Agrobacterium mediated transformation of maize is performed essentially as described by Zhao (WO 1998/32326). Briefly, immature embryos are isolated from maize and the embryos contacted with a suspension of Agrobacterium containing a T-DNA, where the bacteria are capable of transferring the nucleotide sequence of interest to at least one cell of at least one of the immature embryos.

Step 1: Infection Step. In this step the immature embryos are immersed in an Agrobacterium suspension for the initiation of inoculation.

Step 2: Co-cultivation Step. The embryos are co-cultured for a time with the Agrobacterium.

Step 3: Resting Step. Optionally, following co-cultivation, a resting step may be performed. The immature embryos are cultured on solid medium with antibiotic, but without a selecting agent, for elimination of Agrobacterium and for a resting phase for the infected cells.

Step 4: Selection Step. Inoculated embryos are cultured on medium containing a selective agent and growing transformed callus is recovered. The immature embryos are cultured on solid medium with a selective agent resulting in the selective growth of transformed cells.

Step 5: Regeneration Step. Calli grown on selective medium are cultured on solid medium to regenerate the plants.

ii. Particle Bombardment of Maize

Immature maize embryos are bombarded with a DNA construct comprising the polynucleotide of interest. The construct may also contain the selectable marker gene PAT (Wohlleben, et al., (1988) Gene 70:25-37) that confers resistance to the herbicide Bialaphos. Transformation is performed as follows.

Preparation of Target Tissue:

The ears are surface sterilized in 30% chlorox bleach plus 0.5% Micro detergent for 20 minutes and rinsed two times with sterile water. The immature embryos are excised, placed embryo axis side down (scutellum side up), 25 embryos per plate, on 560Y medium for 4 hours and then aligned within the 2.5-cm target zone in preparation for bombardment.

Preparation of DNA:

The DNA is precipitated onto 0.6 μm (average diameter) gold pellets using a CaCl₂ precipitation procedure as follows: 100 μl prepared gold particles in water; 10 μl (1 μg) DNA in TrisEDTA buffer (1 μg total); 100 μl 2.5 M CaCl₂ and 10 μl 0.1 M spermidine.

Each reagent is added sequentially to the gold particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 μl 100% ethanol and centrifuged for 30 seconds. After the liquid is removed, 105 μl 100% ethanol is added to the final gold particle pellet. For particle gun bombardment, the gold/DNA particles are briefly sonicated and 10 μl spotted onto the center of each macrocarrier and allowed to dry about 2 minutes before bombardment.

The sample plates of target embryos are bombarded using approximately 0.1 μg of DNA per shot using the Bio-Rad PDS-1000/He device (Bio-Rad Laboratories, Hercules, Calif.) with a rupture pressure of 650 PSI, a vacuum pressure of 27-28 inches of Hg and a particle flight distance of 8.5 cm. Ten aliquots are taken from each tube of prepared particles/DNA.

Following bombardment, the embryos are kept on 560Y medium for 2 days, then transferred to 560R selection medium containing 3 mg/L Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity.

Medium 560Y comprises 4.0 g/L N6 basal salts (SIGMA C-1416), 1.0 ml/L Eriksson's Vitamin Mix (1000× SIGMA-1511), 0.5 mg/L thiamine HCl, 120 g/L sucrose, 1.0 mg/L 2,4-D and 2.88 g/L L-proline (brought to volume with D-I H₂O following adjustment to pH 5.8 with KOH); 2.0 g/L Gelrite® (added after bringing to volume with D-I H₂O) and 8.5 mg/L silver nitrate (added after sterilizing the medium and cooling to room temperature).

Medium 560R comprises 4.0 g/L N6 basal salts (SIGMA C-1416), 1.0 ml/L Eriksson's Vitamin Mix (1000× SIGMA-1511), 0.5 mg/L thiamine HCl, 30.0 g/L sucrose, and 2.0 mg/L 2,4-D (brought to volume with D-I H₂O following adjustment to pH 5.8 with KOH); 3.0 g/L Gelrite® (added after bringing to volume with D-I H₂O) and 0.85 mg/L silver nitrate and 3.0 mg/L bialaphos (both added after sterilizing the medium and cooling to room temperature).

Medium 288J comprises: 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl and 0.40 g/L glycine brought to volume with D-I H₂O) (Murashige and Skoog, (1962) Physiol Plant 15:473), 100 mg/L myo-inositol, 0.5 mg/L zeatin, 60 g/L sucrose and 1.0 ml/L of 0.1 mM abscissic acid (brought to volume with D-I H₂O after adjusting to pH 5.6); 3.0 g/L Gelrite® (added after bringing to volume with D-I H₂O) and 1.0 mg/L indoleacetic acid and 3.0 mg/L bialaphos (added after sterilizing the medium and cooling to 60° C.).

Medium 272V comprises: 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl and 0.40 g/L glycine brought to volume with D-I H₂O), 0.1 g/L myo-inositol and 40.0 g/L sucrose (brought to volume with D-I H₂O after adjusting pH to 5.6) and 6 g/L Bacto™-agar (added after bringing to volume with D-I H₂O), sterilized and cooled to 60° C.

iii. Particle Bombardment of Soybean

A polynucleotide of interest can be introduced into embryogenic suspension cultures of soybean by particle bombardment using essentially the methods described in Parrott, et al., (1989) Plant Cell Rep 7:615-617. This method, with modifications, is described below.

Seed is removed from pods when the cotyledons are between 3 and 5 mm in length. The seeds are sterilized in a bleach solution (0.5%) for 15 minutes after which time the seeds are rinsed with sterile distilled water. The immature cotyledons are excised by first cutting away the portion of the seed that contains the embryo axis. The cotyledons are then removed from the seed coat by gently pushing the distal end of the seed with the blunt end of the scalpel blade. The cotyledons are then placed in petri dishes (flat side up) with SB1 initiation medium (MS salts, B5 vitamins, 20 mg/L 2,4-D, 31.5 g/L sucrose, 8 g/L TC Agar, pH 5.8). The petri plates are incubated in the light (16 hr day; 75-80 pE) at 26° C. After 4 weeks of incubation the cotyledons are transferred to fresh SB1 medium. After an additional two weeks, globular stage somatic embryos that exhibit proliferative areas are excised and transferred to FN Lite liquid medium (Samoylov, et al., (1998) In Vitro Cell Dev Biol Plant 34:8-13). About 10 to 12 small clusters of somatic embryos are placed in 250 ml flasks containing 35 ml of SB172 medium. The soybean embryogenic suspension cultures are maintained in 35 mL liquid media on a rotary shaker, 150 rpm, at 26° C. with fluorescent lights (20 pE) on a 16:8 hour day/night schedule. Cultures are sub-cultured every two weeks by inoculating approximately 35 mg of tissue into 35 mL of liquid medium.

Soybean embryogenic suspension cultures are then transformed using particle gun bombardment (Klein, et al., (1987) Nature 327:70; U.S. Pat. No. 4,945,050). A BioRad Biolisticä PDS1000/HE instrument can be used for these transformations. A selectable marker gene, which is used to facilitate soybean transformation, is a chimeric gene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. To 50 μL of a 60 mg/mL 1 μm gold particle suspension is added (in order): 5 μL DNA (1 μg/μL), 20 μl spermidine (0.1 M) and 50 μL CaCl₂ (2.5 M). The particle preparation is agitated for three minutes, spun in a microfuge for 10 seconds and the supernatant removed. The DNA-coated particles are washed once in 400 μL 70% ethanol then resuspended in 40 μL of anhydrous ethanol. The DNA/particle suspension is sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. Membrane rupture pressure is set at 1100 psi and the chamber is evacuated to a vacuum of 28 inches mercury. The tissue is placed approximately 8 cm away from the retaining screen, and is bombarded three times. Following bombardment, the tissue is divided in half and placed back into 35 ml of FN Lite medium.

Five to seven days after bombardment, the liquid medium is exchanged with fresh medium. Eleven days post bombardment the medium is exchanged with fresh medium containing 50 mg/mL hygromycin. This selective medium is refreshed weekly. Seven to eight weeks post bombardment, green transformed tissue will be observed growing from untransformed, necrotic embryogenic clusters. Isolated green tissue is removed and inoculated into individual flasks to generate new, clonally propagated, transformed embryogenic suspension cultures. Each new line is treated as an independent transformation event. These suspensions are then subcultured and maintained as clusters of immature embryos or tissue is regenerated into whole plants by maturation and germination of individual embryos.

### Example 7

### DNA Isolation from Callus and Leaf Tissues

Putative transformation events can be screened for the presence of the transgene. Genomic DNA is extracted from calli or leaves using a modification of the CTAB (cetyltriethylammonium bromide, Sigma H5882) method described by Stacey and Isaac, (1994 In Methods in Molecular Biology 28:9-15, Ed. Isaac, Humana Press, Totowa, N.J.). Approximately 100-200 mg of frozen tissue is ground into powder in liquid nitrogen and homogenized in 1 ml of CTAB extraction buffer (2% CTAB, 0.02 M EDTA, 0.1 M TrisHCl pH 8, 1.4 M NaCl, 25 mM DTT) for 30 min at 65° C. Homogenized samples are allowed to cool at room temperature for 15 min before a single protein extraction with approximately 1 ml 24:1 v/v chloroform:octanol is done. Samples are centrifuged for 7 min at 13,000 rpm and the upper layer of supernatant collected using wide-mouthed pipette tips. DNA is precipitated from the supernatant by incubation in 95% ethanol on ice for 1 hr. DNA threads are spooled onto a glass hook, washed in 75% ethanol containing 0.2 M sodium acetate for 10 min, air-dried for 5 min and resuspended in TE buffer. Five μl RNAse A is added to the samples and incubated at 37° C. for 1 hr. For quantification of genomic DNA, gel electrophoresis is performed using a 0.8% agarose gel in 1×TBE buffer. One microlitre of each of the samples is fractionated alongside 200, 400, 600 and 800 ng μl-1λ uncut DNA markers.

That which is claimed:

1. An isolated nucleic acid molecule comprising a promoter polynucleotide comprising a nucleotide sequence selected from the group consisting of:
(a) a nucleotide sequence comprising the nucleotide sequence of SEQ ID NO: 34;
(b) a nucleotide sequence comprising at least 50 consecutive nucleotides of SEQ ID NO: 34, wherein the nucleotide sequence initiates transcription in a plant cell; and
(c) a nucleotide sequence having at least 80% sequence identity to the nucleotide sequence set forth in SEQ ID NO: 34, wherein the nucleotide sequence initiates transcription in a plant cell.

2. The isolated nucleic acid molecule of claim 1, wherein the promoter polynucleotide initiates transcription in an egg cell-preferred or embryonic cell-preferred manner.

3. An expression cassette comprising the nucleic acid molecule of claim 1 or 2 operably linked to a heterologous polynucleotide of interest.

4. A vector comprising the expression cassette of claim 3.

5. A plant cell comprising the expression cassette of claim 3.

6. The plant cell of claim 5, wherein said expression cassette is stably integrated into the genome of the plant cell.

7. The plant cell of claim 5, wherein said plant cell is from a monocot.

8. The plant cell of claim 7, wherein said monocot is maize.

9. A plant comprising the expression cassette of claim 3.

10. The plant of claim 9, wherein said plant is a monocot.

11. The plant of claim 10, wherein said monocot is selected from the group comprising: maize, wheat, rice, barley, sorghum, millet, sugarcane and rye.

12. The plant cell of claim 5, wherein said plant cell is from a dicot.

13. The plant cell of claim 7, wherein said dicot is selected from the group comprising: soy, Brassica sp., cotton, safflower, tobacco, alfalfa and sunflower.

14. The plant of claim 9, wherein said plant is a dicot.

15. The plant of claim 10, wherein said dicot is selected from the group comprising: soy, Brassica sp., cotton, safflower, tobacco, alfalfa and sunflower.

16. The plant of any one of claims 9-15, wherein said expression cassette is stably incorporated into the genome of the plant.

17. The plant of any one of claims 9-15, wherein said heterologous polynucleotide of interest encodes a reporter gene product.

18. The plant of claim 17, wherein said reporter gene product encodes a fluorophore.

19. The plant of claim 18, wherein said fluorophore is selected from the group comprising: DS-RED, ZS-GREEN, ZS-YELLOW, and AM-CYAN, AC-GFP, eGFP, eCFP. eYFP, eBFP, a “fruit” fluoorescent protein (UC system); tagRFP, tagBFP, mKate, mKate2, tagYFP, tagCFP, tagGFP, TurboGFP2, TurboYFP, TurboRFP, TurboFP602, TurboFP635, TurboFP650, NirFP or Cerulean.

20. The plant of any one of claims 9-15 wherein said heterologous polynucleotide of interest encodes a gene product that is involved in organ development, stem cell development, cell growth stimulation, organogenesis, somatic embryogenesis initiation, adventitious embryony initiation, egg cell specification, self-reproducing plants or development of the apical meristem.

21. The plant of claim 20 wherein said gene product is selected from the group consisting of: WUS, CLAVATA, Babyboom, LEC (leafy cotyledon), MYB115, Embryomaker, RKD family genes and MYB118 genes.

22. The plant of any one of claims 9-15, wherein said heterologous polynucleotide of interest alters the phenotype of said plant.

23. The plant of any one of claims 9-15, wherein said heterologous nucleotide of interest encodes a cytotoxin.

24. The plant of claim 23, wherein said cytotoxin comprises an intein coding sequence or a split intein coding sequence.

25. The plant of claim 23 or 24, wherein said cytotoxin is selected from the group including but not limited to: barnase, DAM-methylase, and ADP ribosylase, RNases, nucleases, methylases, membrane pore forming proteins, apoptosis inducing proteins, and ADP-Ribosyltransf erase toxins including but not limited to, PT toxins, C2 toxins, C. difficile transferase, iota toxin, C. spiroforme toxin, DT toxin, LT1, LT2, Tox A and CT toxin.

26. The plant of claim 25, wherein barnase is preferentially expressed in the egg cell.

27. The plant of claim 25 or 26, wherein said plant further expresses barstar.

28. The plant of claim 27, wherein said barstar is expressed constitutively or preferentially expressed in the ovule of said plant.

29. The plant of any one of claims 23-27, wherein expression of said cytotoxin causes ablation of the egg cell.

30. The plant of claim 29, wherein said egg cell ablation results in female sterility.

31. The plant of claim 29 or 30, further comprising a second polynucleotide encoding a RKD transcription factor operably linked to a promoter, wherein said promoter expresses said RKD transcription factor in the ovule tissues of said plant.

32. A transgenic seed of the plant of any one of claims 9-31, wherein the seed comprises said expression cassette.

33. A method for expressing a heterologous polynucleotide of interest in a plant or a plant cell, said method comprising introducing into the plant or the plant cell a expression cassette comprising a promoter polynucleotide operably linked to a heterologous polynucleotide of interest, wherein said promoter polynucleotide comprises a nucleotide sequence selected from the group consisting of:
(a) a nucleotide sequence comprising the nucleotide sequence of SEQ ID NO: 34;
(b) a nucleotide sequence comprising at least 50 consecutive nucleotides of SEQ ID NO: 34, wherein the nucleotide sequence initiates transcription in a plant cell; and
(c) a nucleotide sequence having at least 80% sequence identity to the nucleotide sequence set forth in SEQ ID NO: 34, wherein the nucleotide sequence initiates transcription in a plant cell.

34. The method of claim 33, wherein said expression cassette is stably incorporated into the genome of said plant or plant cell.

35. The method of claim 33 or 34, wherein said heterologous polynucleotide of interest encodes a reporter gene product.

36. The method of claim 35, wherein said reporter gene product encodes a fluorophore.

37. The method of claim 36, wherein said fluorophore is selected from the group consisting of: DS-RED, ZS-GREEN, ZS-YELLOW, AC-GFP, AM-CYAN, and AM-CYAN1, AC-GFP, eGFP, eCFP. eYFP, eBFP, a “fruit” fluorescent protein (UC system); tagRFP, tagBFP, mKate, mKate2, tagYFP, tagCFP, tagGFP, TurboGFP2, TurboYFP, TurboRFP, TurboFP602, TurboFP635, TurboFP650, NirFP or Cerulean.

38. A method for expressing a polynucleotide preferentially in ovule tissues of a plant, said method comprising introducing into a plant cell an expression cassette and regenerating a plant from said plant cell, said plant having stably incorporated into its genome the expression cassette, said expression cassette comprising a promoter polynucleotide operably linked to a heterologous polynucleotide of interest, wherein said promoter polynucleotide comprises a nucleotide sequence selected from the group consisting of:
(a) a nucleotide sequence comprising the nucleotide sequence of SEQ ID NO: 34;
(b) a nucleotide sequence comprising at least 50 consecutive nucleotides of SEQ ID NO: 34; and
(c) a nucleotide sequence having at least 80% sequence identity to the nucleotide sequence set forth in SEQ ID NO: 34,
wherein said promoter polynucleotide preferentially initiates transcription in cell types within the ovule tissues of a plant.

39. The method of claim 38, wherein said cell types are found within the egg sac of an angiosperm.

40. The method of claim 38 or 39, wherein said promoter polynucleotide preferentially initiates transcription in the egg cell or an embryonic cell of a plant ovule.

41. The method of any one of claims 38-40, further comprising detecting said expressed heterologous polynucleotide of interest.

42. The method of any one of claims 38-41, wherein detection of said expressed heterologous polynucleotide of interest identifies the cell type of said ovule tissues or detection of the absence of said expressed heterologous polynucleotide of interest indicates the absence of said cell type.

43. The method of claim 41 or 42, wherein said cell types are detected prior to fertilization.

44. The method of claim 41 or 42, wherein said cell types are detected after fertilization.

45. The method of any one of claims 38-44, wherein detection of said expressed heterologous polynucleotide of interest identifies the cell type of said plant cell as an egg cell or an embryonic cell.

46. The method of any one of claims 38-44, wherein said heterologous polynucleotide of interest encodes a reporter gene product.

47. The method of claim 46, wherein said reporter gene product encodes a fluorophore.

48. The method of claim 47, wherein said fluorophore is selected from the group consisting of: DS-RED, ZS-GREEN, ZS-YELLOW, AC-GFP, AM-CYAN, and AM-CYAN1, AC-GFP, eGFP, eCFP. eYFP, eBFP, a “fruit” fluorescent protein (UC system); tagRFP, tagBFP, mKate, mKate2, tagYFP, tagCFP, tagGFP, TurboGFP2, TurboYFP, TurboRFP, TurboFP602, TurboFP635, TurboFP650, NirFP or Cerulean.

49. The method of any one of claims 33-48, wherein said heterologous nucleotide of interest encodes a cytotoxin.

50. The method of any one of claims 33-48, further comprising introducing into said plant or plant cell a second expression cassette comprising a second promoter polynucleotide operably linked to a second heterologous polynucleotide of interest, wherein said second heterologous polynucleotide of interest encodes a cytotoxin.

51. The method of claim 50, wherein said second promoter polynucleotide comprises a nucleotide sequence selected from the group consisting of:
(a) a nucleotide sequence comprising the nucleotide sequence of SEQ ID NO: 34;
(b) a nucleotide sequence comprising at least 50 consecutive nucleotides of SEQ ID NO: 34; and
(c) a nucleotide sequence having at least 80% sequence identity to the nucleotide sequence set forth in SEQ ID NO: 34,
wherein said promoter polynucleotide initiates transcription in cell types within the ovule tissues of a plant.

52. The method of any one of claims 49-51, wherein said cytotoxin comprises an intein coding sequence or a split intein coding sequence.

53. The method of any one of claims 49-51, wherein said cytotoxin is selected from the group consisting of: barnase, DAM-methylase, and ADP ribosylase.

54. The method of claim 53 wherein barnase is preferentially expressed in the egg cell.

55. The method of claim 53 or 54, wherein said plant further expresses barstar.

56. The method of claim 55 wherein said barstar is expressed constitutively or preferentially expressed in the ovule of said plant.

57. The method of any one of claims 49-56, wherein expression of said cytotoxin results in ablation of the egg cell.

58. The method of claim 57, wherein said egg cell ablation results in female sterility of said plant.

59. The method of claim 57 or 58, wherein at least one synergid is not ablated.
